(19) United States
(12) Patent Application Publication
(10) Pub. No.: US 2024/0110208 A1
(43) Pub. Date: Apr. 4, 2024

(54) GENE EDITING SYSTEM OF CANDIDA VISWANATHII, GENE EDITING METHOD THEREOF, TRANSFORMANT FOR PRODUCING DODECANEDIOIC ACID AND METHOD FOR PRODUCING DODECANEDIOIC ACID

(71) Applicants: NATIONAL TSING HUA UNIVERSITY, HSINCHU (TW); Chang Chun Plastics Co., Ltd., TAIPEI (TW); Chang Chun Petrochemical Co., LTD., TAIPEI (TW); DAIREN CHEMICAL CORP., TAIPEI (TW)

(72) Inventors: Yu-Chen HU, Hsinchu (TW); Nam Ngoc PHAM, Hsinchu (TW); June-Yen CHOU, Taipei City (TW); Hsing-Yun WANG, Taipei City (TW); Vincent Jianan LIU, Taipei City (TW)

(21) Appl. No.: 18/189,272

(22) Filed: Mar. 24, 2023

(30) Foreign Application Priority Data
Sep. 29, 2022 (TW) 111137112

(51) Int. Cl. C12P 7/6409; C12N 15/52; C12N 15/90

(52) U.S. Cl. CPC C12P 7/6409; C12N 15/52; C12N 15/905; C12Y 101/01; C12Y 101/0302; C12Y 114/14; C12Y 207/01086

(57) ABSTRACT

A gene editing system of Candida viswanathii includes a Candida viswanathii, a first gene editing fragment and a second gene editing fragment. The first gene editing fragment successively includes a first homology arm and a screening gene. The second gene editing fragment is connected to a C-terminus of the first gene editing fragment and includes a second homology arm, a Cas9 expression cassette and a sgRNA cassette. The Cas9 expression cassette successively includes a Cas9 promoter, a Cas9 gene and three nuclear localization sequences. The sgRNA cassette successively includes a sgRNA promoter, a first ribozyme, a targeting sequence, a scaffold and a second ribozyme. The first gene editing fragment and the second gene editing fragment are constructed as a linear fragment for gene editing of a chromosome of the Candida viswanathii.

### RELATED APPLICATIONS

This application claims priority to Taiwan Application Serial Number 111137112, filed Sep. 29, 2022, which is herein incorporated by reference.

### SEQUENCE LISTING XML

A sequence listing XML submitted as an xml file via EFS-WEB is incorporated herein by reference. The sequence listing XML file submitted via EFS-WEB with the name “CP-5702-US SEQ LIST” was created on Mar. 21, 2023, which is 101,547 bytes in size.

### BACKGROUND

### Technical Field

The present disclosure relates to a gene editing technology of a microorganism. More particularly, the present disclosure relates to a gene editing system of Candida viswanathii and uses thereof.

### Description of Related Art

Candida viswanathii is a budding yeast that can spontaneously convert long-chain alkanes to the corresponding acids/diacids and consume them as carbon sources for growth via the β-oxidation pathway. Gene-edited strains improved by metabolic engineering are promising cell factories for industrial applications. For example, enzymes involved in related oxidative metabolic pathways can be blocked, while the key enzymes that convert alkanes to acids/diacids can be retained and/or enhanced, so that the gene-engineered strains can consume alkanes stably and continuously, and use the carbon flux in the body for the production of acid/diacid chemicals.

However, most of the current gene engineering of Candida viswanathii is performed by high-pressure selective homologous recombination, which is not only has a low probability of recombination, but also can only edit one gene in one gene editing process. The high-pressure selective homologous recombination is very limited by the size and editing site of the deleted/inserted edited fragment, which is laborious and time-consuming. In addition, Candida viswanathii is one of Candida species having diploid genomes with multiple homologous chromosomes. Due to the characteristic of the parasexual cycle, Candida viswanathii may not undergo meiosis during reproduction, but gradually lose some chromosomes in multiple mitosis and then restore to the original ploidy, resulting in deletion or recombination of edited fragments, greatly increasing the instability of the strain and operational difficulty of gene editing engineering.

CRISPR-Cas9 gene editing technology has been widely used in various organisms in recent years, which can precisely and seamlessly target editing of chromosomes, overcoming the bottlenecks of editing efficiency and editing technology faced by biology-related fields. Although the CRISPR-Cas9 gene editing technology has excellent cutting ability, DNA sizes of the expression cassette of the Cas9 protein and the sgRNA used to guide the cleavage of the Cas9 protein are quite large. In addition, the DNA size of the entire editing system also will be too large due to the insertion of the expression cassettes of other exogenous genes that need to be integrated, which will affect the editing effect. Although the CRISPR-Cas9 system has been available for many years and has been improved many times, it has not yet been successfully established in Candida viswanathii. Therefore, establishing a gene editing technology in Candida viswanathii that can perform gene editing with high efficiency and enable the edited target to stably exist in a transformant is an important issue faced by fields at present.

### SUMMARY

According to one aspect of the present disclosure, a gene editing system of Candida viswanathii includes a Candida viswanathii, a first gene editing fragment and a second gene editing fragment. The first gene editing fragment successively includes a first homology arm and a screening gene. The second gene editing fragment is connected to a C-terminus of the first gene editing fragment, and successively includes a second homology arm, a Cas9 expression cassette and a sgRNA cassette. The Cas9 expression cassette successively includes a Cas9 promoter, a Cas9 gene and three nuclear localization sequences. The sgRNA cassette successively includes a sgRNA promoter, a first ribozyme, a targeting sequence, a scaffold and a second ribozyme. The first gene editing fragment and the second gene editing fragment are constructed as a linear fragment for gene editing of a chromosome of the Candida viswanathii. The first homology arm and the second homology arm respectively correspond to a specific fragment of a gene on the chromosome of the Candida viswanathii, and the targeting sequence corresponds to a specific sequence of the gene on the chromosome of the Candida viswanathii.

According to another aspect of the present disclosure, a gene editing method of Candida viswanathii includes steps as follows. A first gene editing fragment is constructed, wherein the first gene editing fragment successively includes a first homology arm and a screening gene. A second gene editing fragment is constructed, wherein the second gene editing fragment is connected to a C-terminus of the first gene editing fragment, and successively includes a second homology arm, a Cas9 expression cassette and a sgRNA cassette. The Cas9 expression cassette successively includes a Cas9 promoter, a Cas9 gene and three nuclear localization sequences. The sgRNA cassette successively includes a sgRNA promoter, a first ribozyme, a targeting sequence, a scaffold and a second ribozyme. The first gene editing fragment and the second gene editing fragment are constructed as a linear fragment for gene editing of a chromosome of a Candida viswanathii. The first homology arm and the second homology arm respectively correspond to a specific fragment of a gene on the chromosome of the Candida viswanathii, and the targeting sequence corresponds to a specific sequence of the gene on the chromosome of the Candida viswanathii. A transformation step is performed, wherein the first gene editing fragment and the second gene editing fragment are transformed into the Candida viswanathii to obtain a transformant. A transformant cultivation step is performed, wherein the transformant is cultivated in a selection medium at an editing temperature for an editing time, the Cas9 expression cassette expresses the Cas9 gene, the sgRNA cassette expresses the targeting sequence, the first homology arm and the second homology arm are respectively homologously recombined with the specific fragment, and the first gene editing fragment and the second gene editing fragment located between the first homology arm and the second homology arm are integrated into the gene of the transformant.

According to still another aspect of the present disclosure, a transformant for producing dodecanedioic acid includes a host cell and at least two exogenous genes. The host cell is Candida viswanathii. The at least two exogenous genes includes CYP52A19 gene and CPRb gene, and the at least two exogenous genes are integrated into a chromosome of the host cell by the gene editing system of Candida viswanathii according to the aforementioned aspect.

According to yet another aspect of the present disclosure, a method for producing dodecanedioic acid includes steps as follows. A reaction substrate is provided, wherein the reaction substrate includes dodecane. A fermentation step is performed, wherein the reaction substrate is inoculated with the transformant for producing dodecanedioic acid according to the aforementioned aspect, and then is cultured at a fermentation condition for a fermentation time to obtain a fermented substance, and the fermented substance includes dodecanedioic acid.

### BRIEF DESCRIPTION OF THE DRAWINGS

The present disclosure can be more fully understood by reading the following detailed description of the embodiment, with reference made to the accompanying drawings as follows:

FIG. 1 is a schematic view showing a linear fragment of a gene editing system of Candida viswanathii according to one embodiment of one aspect of the present disclosure.

FIG. 2A is a schematic view showing a first gene editing fragment and a second gene editing fragment of a gene editing system of Candida viswanathii according to another embodiment of one aspect of the present disclosure.

FIG. 2B is a schematic view showing a first gene editing fragment and a second gene editing fragment of a gene editing system of Candida viswanathii according to still another embodiment of one aspect of the present disclosure.

FIG. 3 is a flow diagram showing a gene editing method of Candida viswanathii according to another aspect of the present disclosure.

FIG. 4 is a flow diagram showing a method for producing dodecanedioic acid according to still another aspect of the present disclosure.

FIG. 5A is a schematic view showing constructions of sgRNA cassettes of Example 1 to Example 3 of the present disclosure for screening combinations of a first ribozyme and a second ribozyme.

FIG. 5B shows analytical results of a colony formation of Example 1 to Example 3 of the present disclosure.

FIG. 5C is a quantitative diagram showing a colony phenotype ratio in FIG. 5B.

FIG. 5D shows analytical results of Example 2 of the present disclosure verified by colony PCR.

FIG. 5E shows analytical results of a colony formation of a nutrient deficiency test of Example 2 of the present disclosure.

FIG. 6A shows analytical results of a colony formation for verifying the optimized homologous recombination strategy of the present disclosure.

FIG. 6B is a quantitative diagram showing colony phenotype ratio in FIG. 6A.

FIG. 6C shows analytical results of a colony formation of the optimized homologous recombination strategy of the present disclosure verified by colony PCR.

FIG. 6D shows analytical results of a stability of a transformant of the present disclosure verified by colony PCR.

FIG. 7 shows analytical results of marker-free transformant of the present disclosure verified by colony PCR.

FIG. 8 is a schematic view showing pathway for dodecane conversion to dodecanedioic acid in Candida viswanathii.

FIG. 9A is a schematic view showing constructions of expression cassettes of Example 4 to Example 22 of the present disclosure for screening enzymes producing dodecanedioic acid.

FIG. 9B shows analytical results of titer of dodecanedioic acid from culture supernatants of Example 4 to Example 22 of the present disclosure cultured in shake flasks.

FIG. 10A is a schematic view showing constructions of Example 23 to Example 27 of the present disclosure for screening integrated site.

FIG. 10B shows analytical results of titer of dodecanedioic acid from culture supernatants of Example 23 to Example 27 of the present disclosure cultured in shake flasks.

FIG. 10C is a schematic view showing constructions of expression cassettes of Example 26 and Example 28 to Example 33 of the present disclosure for screening and optimizing enzymes for producing dodecanedioic acid.

FIG. 10D shows analytical results of titer of dodecanedioic acid from culture supernatants of Example 26 and Example 28 to Example 33 of the present disclosure cultured in shake flasks.

FIG. 10E shows analytical results of titer of dodecanedioic acid by fermentation production of Candida viswanathii.

FIG. 10F shows analytical results of titer of dodecanedioic acid by fermentation production of P-19C strain of the present disclosure.

FIG. 11A is a schematic view showing constructions of expression cassettes of Example 34 to Example 36 of the present disclosure for screening enzymes of the long-chain alcohol oxidation pathway.

FIG. 11B is a schematic view showing the gene editing system of Candida viswanathii of the present disclosure constructing Example 34 to Example 36 in Candida viswanathii.

FIG. 11C shows analytical results of titer of dodecanedioic acid from culture supernatants of Example 34 to Example 36 of the present disclosure cultured in shake flasks.

FIG. 11D shows analytical results of titer of dodecanedioic acid by fermentation production of PF19C strain of the present disclosure.

FIG. 12A is a schematic view showing constructions of expression cassettes of Example 37 to Example 41 of the present disclosure for screening enzymes of nicotinamide adenine di nucleotide phosphate (NADPH)-regenerating pathway.

FIG. 12B shows analytical results of titer of dodecanedioic acid from culture supernatants of Example 37 to Example 41 of the present disclosure cultured in shake flasks.

FIG. 13A is a schematic view showing construction of PFP19C strain of the present disclosure.

FIG. 13B is a schematic view showing the edited chromosome of the PFP19C strain of the present disclosure.

FIG. 13C shows analytical results of titer of dodecanedioic acid by fermentation production of PFP19C strain of the present disclosure.

### DETAILED DESCRIPTION

### Gene Editing System of Candida viswanathii

A gene editing system of Candida viswanathii of the present disclosure includes a Candida viswanathii, a first gene editing fragment 110 and a second gene editing fragment 120. The second gene editing fragment 120 is connected to a C-terminus of the first gene editing fragment 110, and the first gene editing fragment 110 and the second gene editing fragment 120 are constructed as a linear fragment 100 for gene editing of a chromosome of the Candida viswanathii.

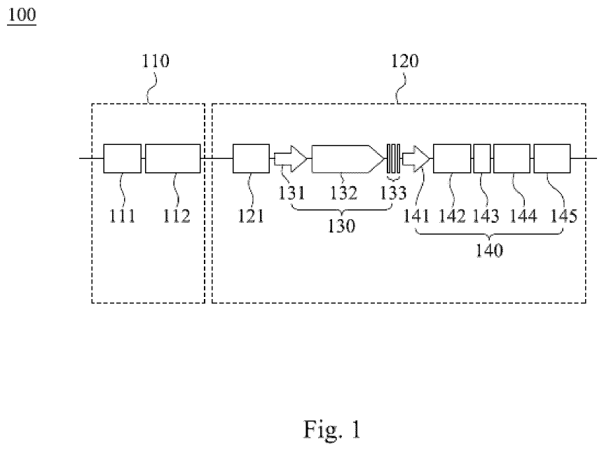

Reference is made to FIG. 1, which is a schematic view showing a linear fragment 100 of the gene editing system of Candida viswanathii according to one embodiment of one aspect of the present disclosure. In FIG. 1, the linear fragment 100 includes the first gene editing fragment 110 and the second gene editing fragment 120. The first gene editing fragment 110 successively includes a first homology arm 111 and a screening gene 112. The second gene editing fragment 120 is connected to a C-terminus of the first gene editing fragment 110, and the second gene editing fragment 120 successively includes a second homology arm 121, a Cas9 expression cassette 130 and a sgRNA cassette 140. The Cas9 expression cassette 130 successively includes a Cas9 promoter 131, a Cas9 gene 132 and three nuclear localization sequences 133. The sgRNA cassette 140 successively includes a sgRNA promoter 141, a first ribozyme 142, a targeting sequence 143, a scaffold 144 and a second ribozyme 145. The first gene editing fragment 110 and the second gene editing fragment 120 are constructed as a linear fragment 100 for gene editing of the chromosome of the Candida viswanathii. The first homology arm 111 and the second homology arm 121 respectively correspond to a specific fragment of a gene on the chromosome of the Candida viswanathii, and the targeting sequence 143 corresponds to a specific sequence of the gene on the chromosome of the Candida viswanathii.

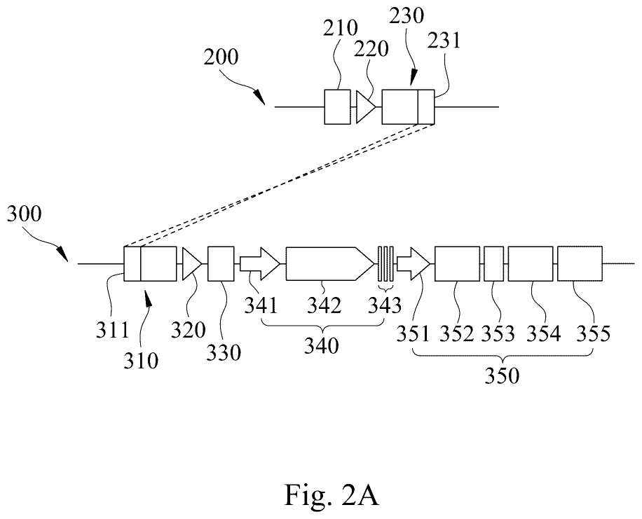

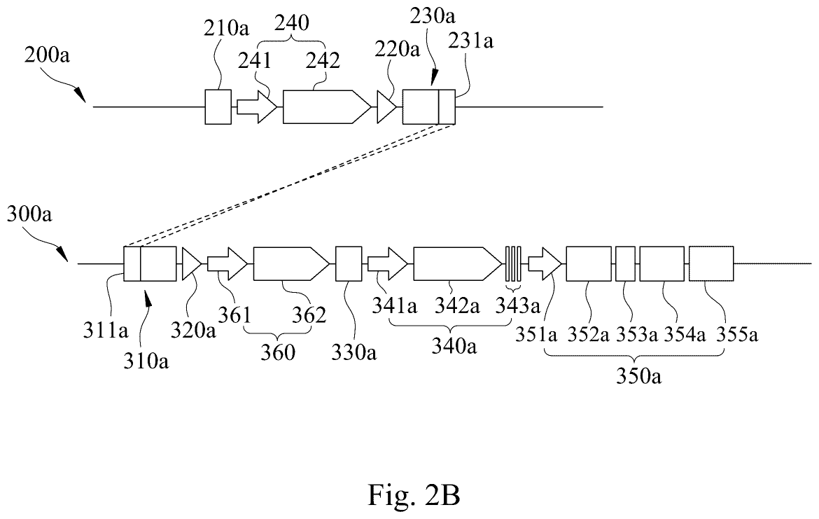

Reference is made to FIG. 2A, which is a schematic view showing a first gene editing fragment 200 and a second gene editing fragment 300 of a gene editing system of Candida viswanathii according to another embodiment of one aspect of the present disclosure. The first gene editing fragment 200 successively includes a first homology arm 210 and a first screening gene fragment 230 of a screening gene (not numbered). The second gene editing fragment 300 successively includes a second screening gene fragment 310 of the screening gene, a second homology arm 330, a Cas9 expression cassette 340 and a sgRNA cassette 350. The Cas9 expression cassette 340 successively includes a Cas9 promoter 341, a Cas9 gene 342 and three nuclear localization sequences 343. The sgRNA cassette 350 successively includes a sgRNA promoter 351, a first ribozyme 352, a targeting sequence 353, a scaffold 354 and a second ribozyme 355. The first gene editing fragment 200 and the second gene editing fragment 300 are constructed as a linear fragment (not numbered) for gene editing of the chromosome of the Candida viswanathii. The first homology arm 210 and the second homology arm 330 respectively correspond to a specific fragment of a gene on the chromosome of the Candida viswanathii, and the targeting sequence 353 corresponds to a specific sequence of the gene on the chromosome of the Candida viswanathii.

Specifically, the screening gene (not numbered) of the first gene editing fragment 200 further includes the first screening gene fragment 230. The second gene editing fragment 300 further includes the second screening gene fragment 310 at an N-terminus of the second homology arm 330. The first screening gene fragment 230 has a homologous fragment 231, and the second screening gene fragment 310 has a homologous fragment 311. The first gene editing fragment 200 and the second gene editing fragment 300 are recombined into the linear fragment by the homologous fragment 231 and the homologous fragment 311.

In greater detail, the homologous fragment 231 and the homologous fragment 311 can be a homologous sequence with a size of about 400 bp, thereby improving the success rate and accuracy of recombination between the first gene editing fragment 200 and the second gene editing fragment 300, but the present disclosure is not limited thereto. The sgRNA promoter 351 and the Cas9 promoter 341 can be TDH1 promoter (P_{TDH1}), PGK1 promoter (P_{PGK1}), ACT1 promoter (P_{ACT1}) or ADH1 promoter (P_{ADH1}). Preferably, sgRNA promoter 351 can be P_{TDH1}, and the Cas9 promoter 341 can be P_{PGK1}, but the present disclosure is not limited thereto. The first ribozyme 352 can be Hammerhead (Ham) or tRNA^{Ala} (derived from Candida parapsilosis). Preferably, the first ribozyme 352 can be tRNA^{Ala}, but the present disclosure is not limited thereto. The second ribozyme 355 can be selected from hepatitis delta virus (HDV) or tRNA^{Gly} (derived from Saccharomyces cerevisiae). Preferably, the second ribozyme 355 can be HDV, but the disclosure is not limited thereto. The three nuclear localization sequences can be SV40 NLS (nuclear localization signal), but the disclosure is not limited thereto. The screening gene can be an antibiotic resistance gene, preferably, the screening gene can be a nourseothricin resistance (Nrs^{R}) gene, but the present disclosure is not limited thereto.

In addition, the Frt sequence 220 can be inserted upstream of the first screening gene fragment 230, and the Frt sequence 320 can be inserted downstream of the second screening gene fragment 310, so as to facilitate subsequent demarking engineering to delete the screening gene as required. Therefore, a gene editing technology of Candida viswanathii with high-efficiency that is not limited by the gene editing site and the size of the gene editing fragment can be achieved.

Reference is made to FIG. 2B, which is a schematic view showing a first gene editing fragment 200a and a second gene editing fragment 300a of a gene editing system of Candida viswanathii according to still another embodiment of one aspect of the present disclosure. In FIG. 2B, the first gene editing fragment 200a and the second gene editing fragment 300a are similar to the first gene editing fragment 200 and the second gene editing fragment 300. The difference is that the first gene editing fragment 200a includes at least one expression cassette 240, and the second gene editing fragment 300a includes at least one expression cassette 360. Other identical technical details will not be repeated here.

The first gene editing fragment 200a successively includes a first homology arm 210a, the at least one expression cassette 240 and a first screening gene fragment 230a of a screening gene (not numbered). The at least one expression cassette 240 includes an exogenous gene promoter 241 and an exogenous gene 242.

The second gene editing fragment 300a successively includes a second screening gene fragment 310a of the screening gene, the at least one expression cassette 360, a second homology arm 330a, a Cas9 expression cassette 340a and a sgRNA cassette 350a. The at least one expression cassette 360 includes an exogenous gene promoter 361 and an exogenous gene 362. The Cas9 expression cassette 340a successively includes a Cas9 promoter 341a, a Cas9 gene 342a and three nuclear localization sequences 343a. The sgRNA cassette 350a successively includes a sgRNA promoter 351a, a first ribozyme 352a, a targeting sequence 353a, a scaffold 354a and a second ribozyme 355a. The first gene editing fragment 200a and the second gene editing fragment 300a are constructed as a linear fragment (not numbered) for gene editing of the chromosome of the Candida viswanathii. The first homology arm 210a and the second homology arm 330a respectively correspond to a specific fragment of a gene on the chromosome of the Candida viswanathii, and the targeting sequence 353a corresponds to a specific sequence of the gene on the chromosome of the Candida viswanathii.

Specifically, the screening gene (not numbered) of the first gene editing fragment 200a further includes the first screening gene fragment 230a. The second gene editing fragment 300a further includes the second screening gene fragment 310a at an N-terminus of the second homology arm 330a. The first screening gene fragment 230a has a homologous fragment 231a, and the second screening gene fragment 310a has a homologous fragment 311a. The first gene editing fragment 200a and the second gene editing fragment 300a are recombined into the linear fragment by the homologous fragment 231a and the homologous fragment 311a.

Although the number of the at least one expression cassette in FIG. 2B is two, and they are respectively located in the first gene editing fragment 200a and the second gene editing fragment 300a. The number and location of the at least one expression cassette can be adjusted according to needs of editing, and the disclosure is not limited thereto.

In addition, a Frt sequence 220a can be inserted upstream of the first screening gene fragment 230a, and the a Frt sequence 320a can be inserted downstream of the second screening gene fragment 310a, so as to facilitate subsequent demarking engineering to delete the screening gene as required.

### Gene Editing Method of Candida viswanathii

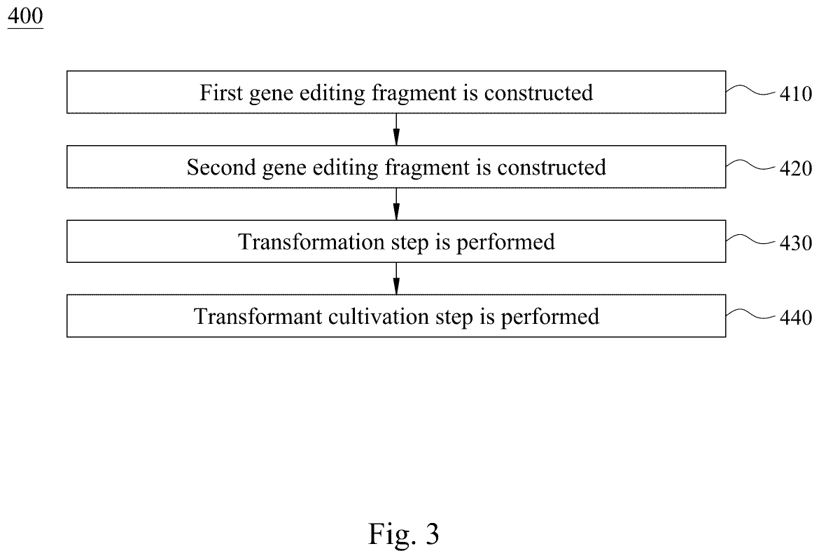

Reference is made to FIG. 3, which is a flow diagram showing a gene editing method of Candida viswanathii 400 according to another aspect of the present disclosure. The gene editing method of Candida viswanathii 400 includes Step 410, Step 420, Step 430 and Step 440.

In Step 410, a first gene editing fragment is constructed. The first gene editing fragment successively includes a first homology arm and a screening gene.

In Step 420, a second gene editing fragment is constructed. The second gene editing fragment is connected to the C-terminus of the first gene editing fragment, and successively includes a second homology arm, a Cas9 expression cassette and a sgRNA cassette. The Cas9 expression cassette successively includes a Cas9 promoter, a Cas9 gene and three nuclear localization sequences. The sgRNA cassette successively includes a sgRNA promoter, a first ribozyme, a targeting sequence, a scaffold and a second ribozyme. The first gene editing fragment and the second gene editing fragment are constructed as a linear fragment for gene editing of a chromosome of a Candida viswanathii. The first homology arm and the second homology arm respectively correspond to a specific fragment of a gene on the chromosome of the Candida viswanathii, and the targeting sequence corresponds to a specific sequence of the gene on the chromosome of the Candida viswanathii.

In greater detail, the first gene editing fragment and/or the second gene editing fragment can include at least one expression cassette, and the at least one expression cassette includes an exogenous gene promoter and an exogenous gene. In practical applications, the number and site of the at least one expression cassette can be adjusted according to editing needs. In addition, a Frt sequence can be inserted into the upstream of the first screening gene fragment and downstream of the second screening gene fragment, so as to facilitate subsequent demarking engineering to delete the screening gene as required.

In Step 430, a transformation step is performed. The first gene editing fragment and the second gene editing fragment are transformed into the Candida viswanathii to obtain a transformant.

In greater detail, when the first homology arm and the second homology arm perform homologous recombination on the specific fragment of Candida viswanathii, a fragment of about 1 kb can be deleted at the same time to improve the efficiency of complete insertion, but the disclosure is not limited thereto.

In Step 440, a transformant cultivation step is performed. The transformant is cultivated in a selection medium at an editing temperature for an editing time, the Cas9 expression cassette expresses the Cas9 gene, the sgRNA cassette expresses the targeting sequence, the first homology arm and the second homology arm are respectively homologously recombined with the specific fragment, and the first gene editing fragment and the second gene editing fragment located between the first homology arm and the second homology arm are integrated into the gene of the transformant.

Specifically, the screening gene of the first gene editing fragment can further include a first screening gene fragment, and the second gene editing fragment can further includes a second screening gene fragment at an N-terminus of the second homology arm. The first screening gene fragment has a homologous fragment, and the second screening gene fragment has a homologous fragment. The first gene editing fragment and the second gene editing fragment are recombined into the linear fragment by the homologous fragment of the first screening gene fragment and the homologous fragment of the second screening gene fragment. That is, the first gene editing fragment and the second gene editing fragment can be the same linear fragment and transformed into the cell of Candida viswanathii for gene editing, or can be recombined after co-transformation into the cell of Candida viswanathii, the timing of recombination can be adjusted according to editing needs, and the disclosure is not limited thereto. Therefore, a gene editing technology of Candida viswanathii with high-efficiency that is not limited by the gene editing site and the size of the gene editing fragment can be achieved.

### Transformant for Producing Dodecanedioic Acid

A transformant for producing dodecanedioic acid of the present disclosure includes a host cell and at least two exogenous genes. The host cell is Candida viswanathii. The at least two exogenous genes includes CYP52A19 gene and CPRb gene, and the at least two exogenous genes are integrated into a chromosome of the host cell by the gene editing system of Candida viswanathii of the present disclosure. Specifically, the at least two exogenous genes are integrated into a chromosome of the host cell by the gene editing system of Candida viswanathii of the present disclosure shown in FIG. 2B.

In greater detail, the at least two exogenous genes can include CYP52A18 gene, FAO2 gene and POS5 gene. Therefore, productivity, purity and molar conversion of dodecanedioic acid can be improved. In addition, the at least two exogenous genes can be integrated into PDX2 gene of the chromosome of the host cell.

### Method for Producing Dodecanedioic Acid

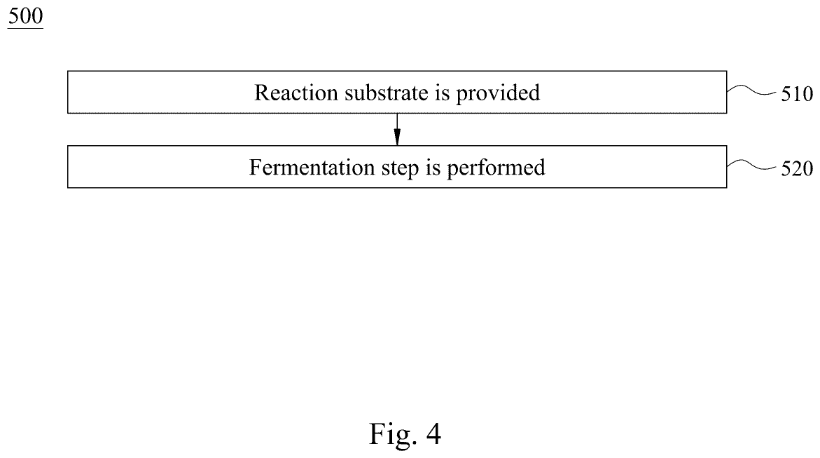

Reference is made to FIG. 4, which is a flow diagram showing a method for producing dodecanedioic acid 500 according to still another aspect of the present disclosure. In FIG. 4, the method for producing dodecanedioic acid 500 include Step 510 and Step 520.

In Step 510, a reaction substrate is provided. The reaction substrate includes dodecane.

In Step 520, a fermentation step is performed. The reaction substrate is inoculated with the transformant for producing dodecanedioic acid according to the aforementioned aspect, and then is cultured at a fermentation condition for a fermentation time to obtain a fermented substance, and the fermented substance includes dodecanedioic acid.

Reference will now be made in detail to the present embodiments of the present disclosure, examples of which are illustrated in the accompanying drawings.

### Example

### I. Assessing the Editing Capacity of the CRISPR-Cas9 System in Candida viswanathii

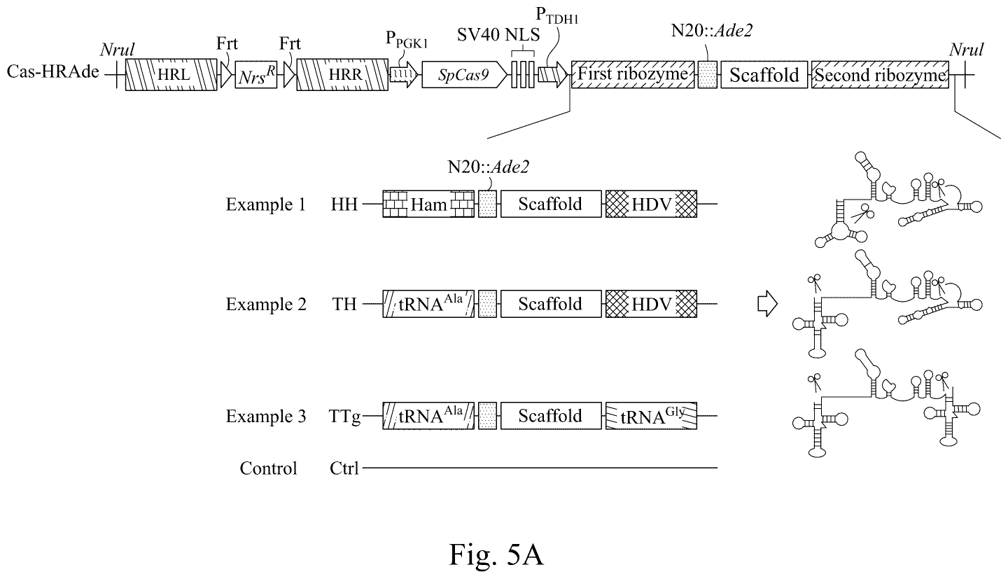

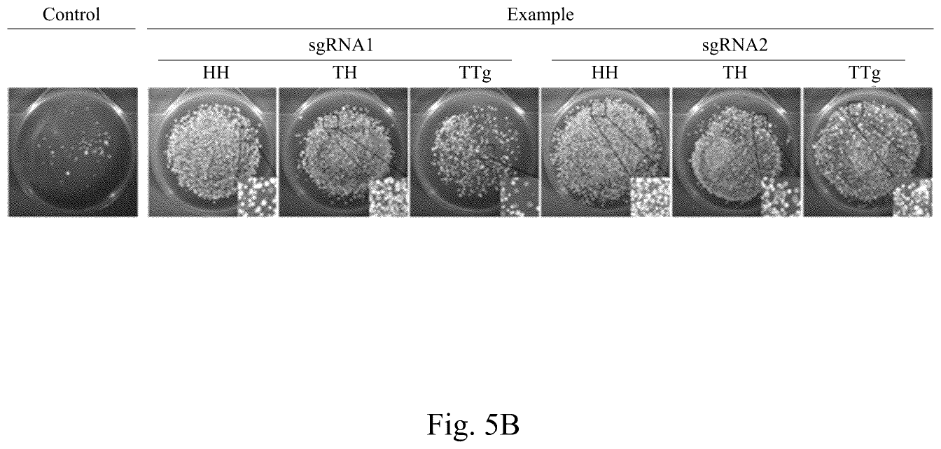

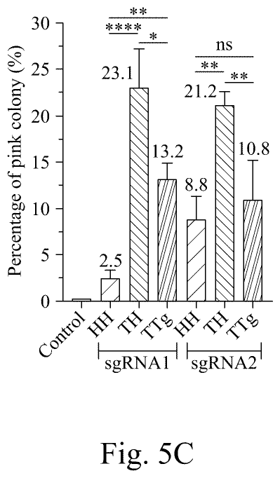

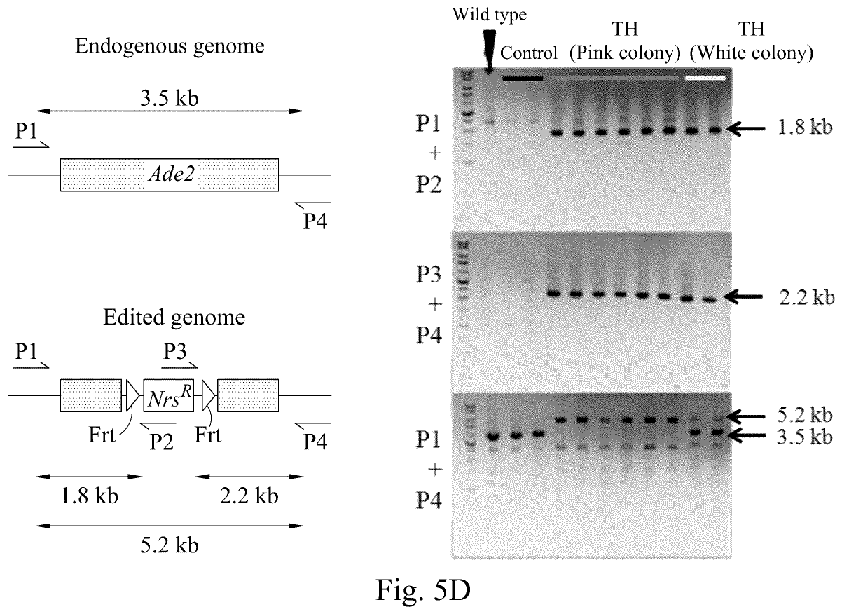

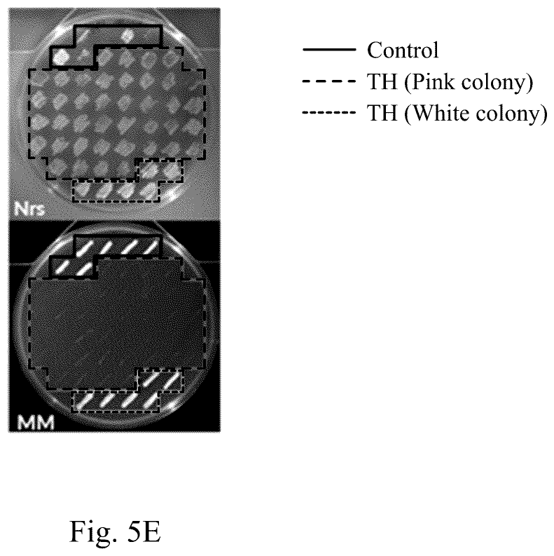

Reference is made to FIG. 5A, which is a schematic view showing constructions of sgRNA cassettes of Example 1 to Example 3 of the present disclosure for screening combinations of a first ribozyme and a second ribozyme. There is no report on the application of CRISPR-Cas9 system to Candida viswanathii, in order to confirm the ability of CRISPR-Cas9 system to integrate exogenous genes into the genome of Candida viswanathii, a linear fragment (represented as Cas-HRAde in FIG. 5A) is constructed experimentally for gene editing ability assessment of CRISPR-Cas9 system to Candida viswanathii. The Cas9 promoter is P_{PGK1} with the sequence referenced as SEQ ID NO: 1 for driving the Cas9 gene (represented as SpCas9 in FIG. 5A) with the sequence referenced as SEQ ID NO: 2 in the Cas9 expression cassette and three nuclear localization sequences (represented as SV40 NLS in FIG. 5A) with the sequence referenced as SEQ ID NO: 3. The sgRNA promoter is P_{TDH1} with the sequence referenced as SEQ ID NO: 4 for driving the sgRNA cassette, and Cas-HRAde includes a Nrs^{R} gene with the sequence referenced as SEQ ID NO: 5 used as a screening gene, and the upstream and downstream of the Nrs^{R} gene are respectively inserted a Frt sequence (represented as Frt in FIG. 5A) with the sequence referenced as SEQ ID NO: 6. The constructed Cas-HRAde is transformed into the cells of wild type Candida viswanathii ATCC 20962 (hereinafter referred as “Candida viswanathii”) by electroporation. A first homology arm (represented as HRL in FIG. 5A) with the sequence referenced as SEQ ID NO: 7 and a second homology arm with the sequence referenced as SEQ ID NO: 8 (represented as HRR in FIG. 5A) are designed to correspond to a specific fragment of the Ade2 gene on the chromosome of Candida viswanathii, and a targeting sequence (represented as N20::Ade2 in FIG. 5A) can correspond to a specific sequence of the Ade2 gene on the chromosome of Candida viswanathii. The Ade2 gene is a reporter gene. When the Nrs^{R} gene is integrated into the Ade2 gene on the chromosome of Candida viswanathii, the colony on the Yeast-Peptone-Dextrose (YPD) plate appears pink. The sgRNA is composed of the targeting sequence (N20::Ade2) and a scaffold with the sequence referenced as SEQ ID NO: 9, and the P_{TDH1} driving the sgRNA cassette is a Pol II promoter, which requires in cis cleavage by the first ribozyme and the second ribozyme on the targeting sequence and the scaffold. Therefore, three different combinations of the first ribozyme expressed upstream of the targeting sequence and the second ribozyme expressed downstream of the scaffold including Hammerhead (Ham) of sequence referenced as SEQ ID NO: 10, hepatitis delta virus (HDV) of sequence referenced as SEQ ID NO: 11, tRNA^{Ala} derived from Candida parapsilosis of sequence referenced as SEQ ID NO: 12 and tRNA^{Gly} derived from Saccharomyces cerevisiae of the sequence referenced as SEQ ID NO: 13 are constructed experimentally as Examples, which are Ham-HDV (Example 1, HH), tRNA^{Ala}-HDV (Example 2, TH) and tRNA^{Ala}-tRNA^{Gly} (Example 3, TTg) respectively. In addition, two sgRNAs, namely sgRNA1 and sgRNA2, are designed in the experiment for targeting different specific sequences on the Ade2 gene, wherein the targeting sequence of sgRNA1 is N20::Ade2-1 of sequence referenced as SEQ ID NO: 14, and the targeting sequence of sgRNA2 is N20::Ade2-2 of sequence referenced as SEQ ID NO: 15. A linear fragment (represented as Ctrl in FIG. 5A) that can only express the SpCas9 gene but has no targeting sequence, scaffold, first ribozyme and second ribozyme is used as a control.

Reference is made to FIG. 5B and FIG. 5C. FIG. 5B shows analytical results of a colony formation of Example 1 to Example 3 of the present disclosure, and FIG. 5C is a quantitative diagram showing a colony phenotype ratio in FIG. 5B. Following nourseothricin (Nrs) selection, Example 1 (HH) to Example 3 (TTg) using sgRNA1 and sgRNA2 yield pink colonies, which indicate that the Nrs^{R} gene can be successfully integrated into the Ade2 gene using the CRISPR-Cas9 system. Specifically, the percentage of pink colony of Example 1 (HH) is about 2.5-8.8%, and the percentage of pink colony of Example 3 (TTg) is about 10.8-13.2%. The percentage of pink colony of Example 2 (TH) is 21.2-23.1%, higher than that of Example 3 (TTg) and Example 1 (HH) at least one time, which indicates that flanking sgRNA with tRNA^{Ala} and HDV as the first ribozyme and the second ribozyme respectively in Example 2 (TH) can more effectively process and cut the sgRNA in Candida viswanathii.

The aforementioned results indicates that the gene editing system of Candida viswanathii of the present disclosure and the use thereof verify that the CRISPR-Cas9 system can indeed edit the genes of Candida viswanathii, and can be modified by adjusting the first ribozyme and the second ribozyme combination to increase the success rate of gene editing.

### II. Optimizing the Editing Capacity of the CRISPR-Cas9 System in Candida viswanathii

Although the CRISPR-Cas9 system has been proven experimentally to be effective in gene editing of Candida viswanathii, there are still some white colonies in the colonies in Example 2 (TH). Therefore, four primers (P1 of the sequence referenced as SEQ ID NO: 16, P2 of the sequence referenced as SEQ ID NO: 17, P3 of the sequence referenced as SEQ ID NO: 18 and P4 of the sequence referenced as SEQ ID NO: 19) are designed to detect the endogenous genome on the chromosome of Candida viswanathii. 6 pink colonies and 2 white colonies are randomly selected, and colony PCR is used to confirm the edited genome of the colonies.

Reference is made to FIG. 5D, which shows analytical results of Example 2 of the present disclosure verified by colony PCR. P1 and P4 are respectively designed on the upstream forward sequence and downstream reverse sequence of the Ade2 gene on the endogenous genome, and P2 and P3 are respectively designed on the front reverse sequence and terminal forward sequence of the Nrs^{R} gene. For the endogenous genome of Candida viswanathii or Control, a PCR product with an expected size of about 3.5 kb can be obtained after PCR with primers P1+P4. For the edited genome successfully integrated the Nrs^{R} gene into the Ade2 gene, a PCR product with an expected size of about 1.8 kb can be obtained after PCR with primers P1+P2, a PCR product with an expected size of about 2.2 kb can be obtained after PCR with primers P3+P4, and a PCR product with an expected size of about 5.2 kb can be obtained after PCR with primers P1+P4. In FIG. 5D, the PCR products of the pink colonies obtained through each set of primers are consistent with the expected size of the PCR products of the edited genome, indicating that the Nrs^{R} gene has been successfully integrated into the Ade2 gene. The PCR products of the white colonies include the expected size of the PCR products of both the endogenous genome and the edited genome, indicating that the white colonies include not only the edited genome that successfully integrated the Nrs^{R} gene into the Ade2 gene, but also the unedited endogenous genome. Reference is made to FIG. 5E, which shows analytical results of a colony formation of a nutrient deficiency test of Example 2 of the present disclosure. White colonies and pink colonies from Example 2 and Control are re-streaked onto the YPD plate containing Nrs (Nrs plate) and the mineral medium plate (MM plate) at the same time. The selected 40 pink colonies all grow well on the Nrs plate but not on the MM plate, while the white colonies from Example 2 grow well on the Nrs plate and the MM plate the same as Control. The results indicate that the pink colonies are homozygous edited transformants (Ade2^{-/-}) that have successfully integrated the Nrs^{R} gene into the two copies of the Ade2 genes of Candida viswanathii, while the white colonies are heterozygous edited transformant (Ade2^{+/-}) that have failed to integrate the Nrs^{R} gene into the two copies of the Ade2 genes of Candida viswanathii.

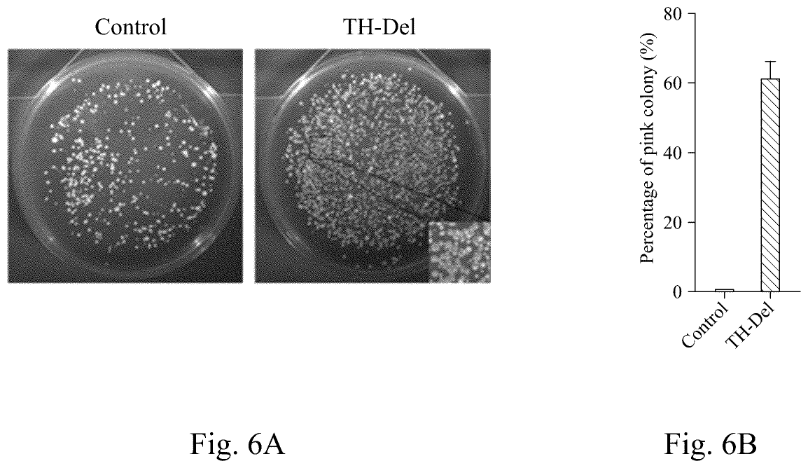

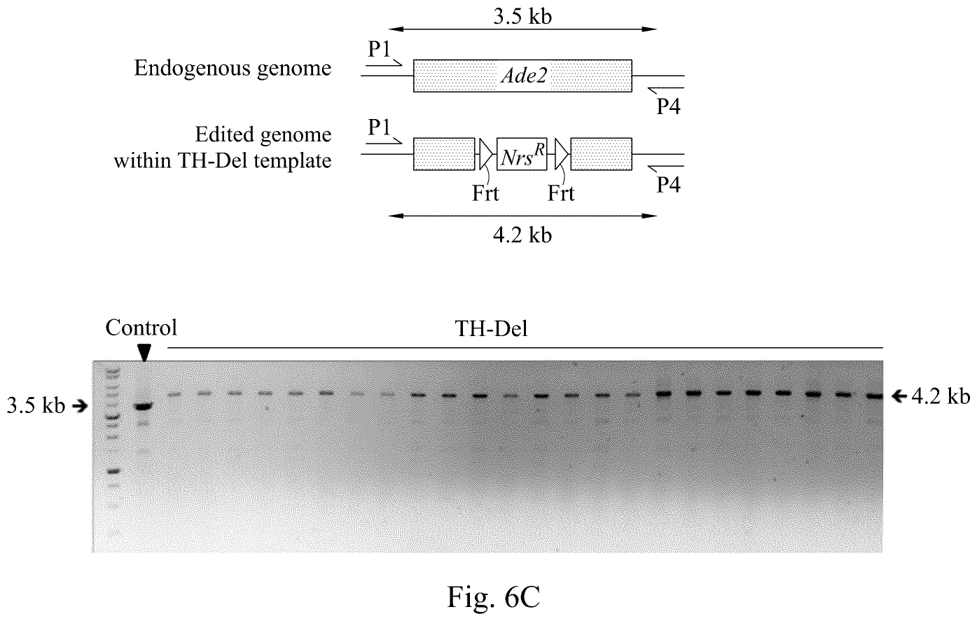

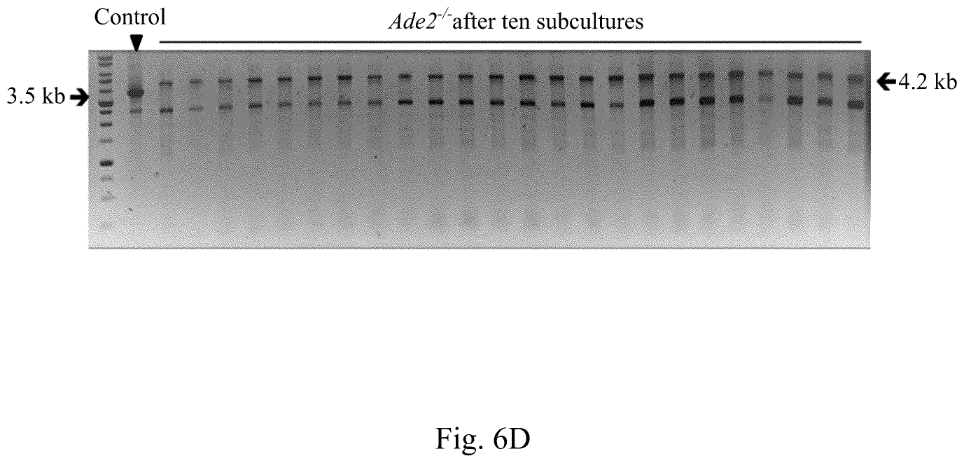

Reference is made to FIG. 6A to 6C. FIG. 6A shows analytical results of a colony formation for verifying the optimized homologous recombination strategy of the present disclosure, FIG. 6B is a quantitative diagram showing colony phenotype ratio in FIG. 6A, and FIG. 6C shows analytical results of a colony formation of the optimized homologous recombination strategy of the present disclosure verified by colony PCR. To enhance homozygous integration frequency, a linear fragment (represented as TH-Del in FIG. 6A to FIG. 6C, and its structure diagram is not show) adjusting the site of the specific fragment on the Ade2 gene corresponding to the first homology arm and the second homology arm is constructed experimentally, and other structures of TH-Del is the same as that of Example 2 (TH). When the first homology arm and the second homology arm undergo homologous recombination with the Ade2 gene through the above-mentioned specific fragment, a fragment of about 1 kb will be deleted to increase the efficiency of integration. The electroporated Candida viswanathii is recovered in 2×YPD medium supplemented with essential amino acids and 40 mg/L adenine under a culture condition (30° C., 250 rpm) to slow down the growth stress and promote stress. After 4 hours recovery, additional 25 mg/L Nrs is added to induce gene editing, and the culture is continued for another 20 hours before streaking the cells onto YPD plates containing Nrs and adenine. The results show that through the above-mentioned optimization of the design of the first homology arm and the second homology arm and the electroporation recovery process, more than 60% of pink colonies Ade2^{-/-} are produced on the Nrs plate, compared to Example 2 (TH) has a nearly three-fold increase in the percentage of pink colony. Furthermore, 24 randomly selected pink colonies edited by TH-Del are performed colony PCR, and all obtained PCR products of 4.2 kb, confirming that their genomes are all homozygous (Ade2^{-/-}). Although Candida viswanathii has polyploid chromosomes, the gene editing system of Candida viswanathii of the present disclosure can successfully integrate the exogenous gene into the target gene and permanently integrate the exogenous gene into the polyploid chromosomes of Candida viswanathii. Therefore, the results indicate that the gene editing system of Candida viswanathii of the present disclosure and uses thereof are highly efficient in completely editing of Candida viswanathii.

Reference is made to FIG. 6D, which shows analytical results of a stability of a transformant of the present disclosure verified by colony PCR. After ten subcultures of the 24 pink colonies Ade2^{-/-}, each colony can still obtain a PCR product of 4.2 kb after colony PCR, which indicates that the gene editing of Candida viswanathii of the present disclosure and the transformant edited by the gene editing of Candida viswanathii of the present disclosure has extremely high genome stability.

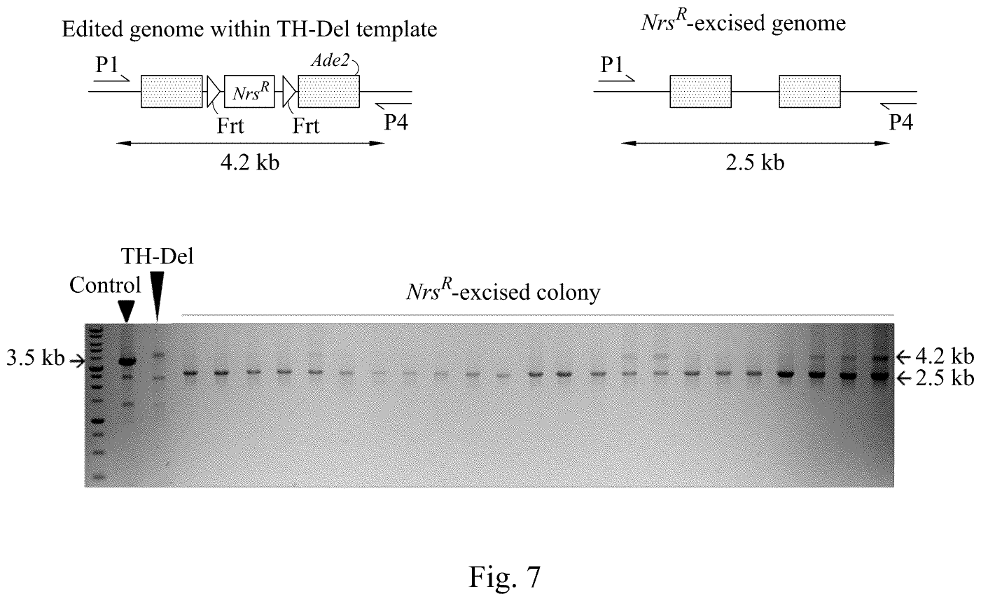

It is worth mentioning that, in order to reduce the cost and considerations required for practical application, such as adding antibiotics to maintain the stability of the strain, replacing the screening gene in response to research and development needs, etc., another plasmid pHyg-Flp (not shown) can be constructed. The plasmid pHyg-Flp includes the Hyg^{R} gene of the sequence referenced as SEQ ID NO: 20 and the Flp gene of the sequence referenced as SEQ ID NO: 21, which can recognize and excise the Frt sequence on the DNA (represented as Frt in FIG. 6C and FIG. 7). Reference is made to FIG. 7, which shows analytical results of marker-free transformant of the present disclosure verified by colony PCR. The results show that after transforming the plasmid pHyg-Flp into Ade2^{+/-} or Ade2^{-/-} and then cultured, all randomly selected colonies obtained a 2.5 kb PCR product confirmed by colony PCR, indicating that the Nrs^{R} gene in the genome had been successfully remove. The results indicate that the CRISPR-Cas9 system established by the gene editing system of Candida viswanathii of the present disclosure can successfully perform marker-free gene editing in Candida viswanathii.

The above results indicate that the gene editing system of Candida viswanathii of the present disclosure and uses thereof not only has the high efficiency of completely editing of Candida viswanathii, but also can use to edit the transformant having extremely high genome stability. Therefore, the gene editing system of Candida viswanathii of the present disclosure also can be used to construct marker-free transformant in response to the needs of practical applications.

### III. Establishing of a Transformant for Producing Dodecanedioic Acid Stably and a Method for Producing Dodecanedioic Acid to Verify the Application Feasibility of the Gene Editing System of Candida viswanathii

### 3.1 Screening of Enzymes for Producing Dodecanedioic Acid

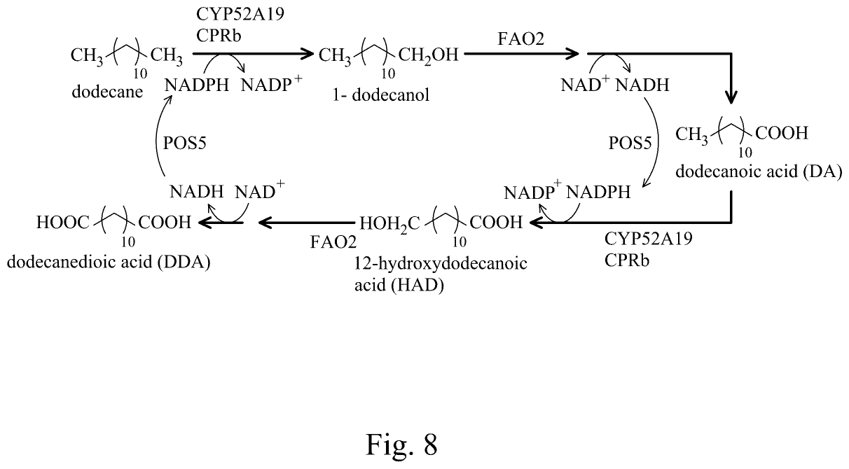

Reference is made to FIG. 8, which is a schematic view showing pathway for dodecane conversion to dodecanedioic acid in Candida viswanathii. Cytochrome P450 monooxygenase (CYP) and nicotinamide adenine dinucleotide phosphate (NADPH) cytochrome reductase (CPR) are a family of enzymes that can catalyze the formation of dicarboxylic acids from various long-chain alkanes, such as CYP52A13, CYP52A15, CYP52A18, CYP52A19 and CPRb. Dodecane can be uptaken into the cell of Candida viswanathii, converted into 1-dodecanol, dodecanoic acid (DA), 12-hydroxydodecanoic acid (HDA) through a series of oxidation pathways, and finally converted to dodecanedioic acid (DDA) by CYP, CPR and other enzymes.

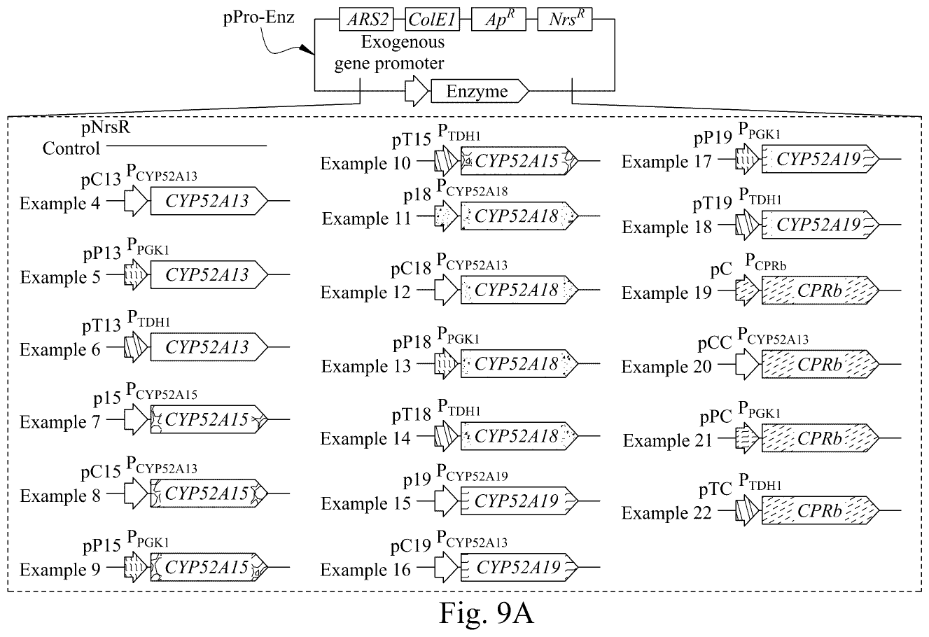

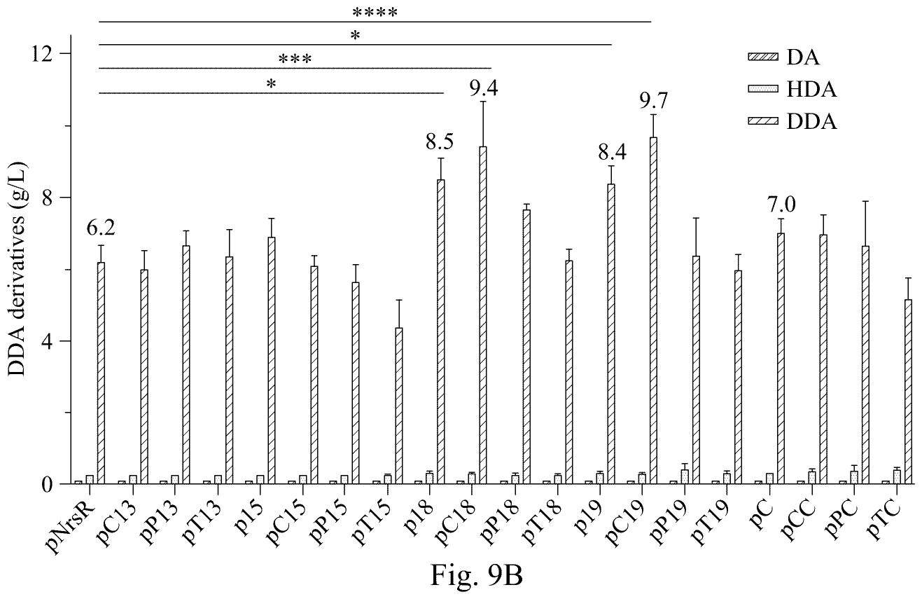

Reference is made to FIG. 9A, which is a schematic view showing constructions of expression cassettes of Example 4 (pC13), Example 5 (pP13), Example 6 (pT13), Example 7 (p15), Example 8 (pC15), Example 9 (pP15), Example 10 (pT15), Example 11 (p18), Example 12 (pC18), Example 13 (pP18), Example 14 (pT18), Example 15 (p19), Example 16 (pC19), Example 17 (pP19), Example 18 (pT19), Example 19 (pC), Example 20 (pCC), Example 21 (pPC) and Example 22 (pTC) of the present disclosure for screening enzymes producing dodecanedioic acid, wherein the dotted line shows the exogenous gene promoter and the exogenous gene of the enzyme producing dodecanedioic acid in the expression cassette of Example 4 to Example 22. In order to construct the transformant for producing dodecanedioic acid stably producing dodecanedioic acid, plasmids pPro-Enz including the Nrs^{R} gene of Example 4 to Example 22 are experimentally constructed. The plasmid pPro-Enz includes the ARS2 gene of the sequence referenced as SEQ ID NO: 22, the ColE1 gene of the sequence referenced as SEQ ID NO: 23, the Ap^{R} gene of the sequence referenced as SEQ ID NO: 24, the Nrs^{R} gene and an expression cassette for screening the enzymes suitable for the conversion of dodecane to DDA in the metabolic pathway of Candida viswanathii. The expression cassette includes an exogenous gene promoter and the exogenous gene of the enzyme to be expressed. The enzyme encoded by the exogenous gene is selected from endogenous CYP or CPRb of Candida viswanathii, such as the CYP52A13 gene of the sequence referenced as SEQ ID NO: 25 (represented as CYP52A13 in FIG. 9A), the CYP52A15 gene of the sequence referenced as SEQ ID NO: 26 (represented as CYP52A15 in FIG. 9A), the CYP52A18 gene of the sequence referenced as SEQ ID NO: 27 (represented as CYP52A18 in FIG. 9A), the CYP52A19 gene of the sequence referenced as SEQ ID NO: 28 (represented as CYP52A19 in FIG. 9A) and the CPRb gene of the sequence referenced as SEQ ID NO: 29 (represented as CPRb in FIG. 9A). The exogenous gene promoter is selected from the endogenous promoter of the above-mentioned endogenous CYP/CPRb (such as PCYP52A15 of sequence referenced as SEQ ID NO: 30, PCYP52A18 of sequence referenced as SEQ ID NO: 31, PCYP52A19 of the sequence referenced as SEQ ID NO: 32 and PCPRb of the sequence referenced as SEQ ID NO: 33), dodecane-inducible promoter (PCYP52A13 of the sequence referenced as SEQ ID NO: 34) or PTDH1 and PPGK1. In addition, a pNrsR that does not express the expression cassette is used as Control. After electroporating the plasmids pPro-Enz of Example 4 to Example 22 into Candida viswanathii respectively, Candida viswanathii containing Example 4 to Example 22 are cultured in shake flasks to produce DDA, and then the titer of DDA in the culture supernatants is analyzed by gas chromatography-flame ionization detector (GC-FID).

Reference is made to FIG. 9B, which shows analytical results of titer of dodecanedioic acid from culture supernatants of Example 4 to Example 22 of the present disclosure cultured in shake flasks. Examples 4 to 22 can produce DDA and only accompanied by a very small amount of by-products HDA and DA. Specifically, the titers of DDA in Example 4 (pC13), Example 5 (pP13), Example 6 (pT13), Example 7 (p15), Example 8 (pC15), Example 9 (pP15) and Example 10 (pT15) expressing the CYP52A13 gene and the CYP52A15 gene are only equivalent to or even lower than that of Control. The titers of DDA in Example 19 (pC), Example 20 (pCC), Example 21 (pPC) and Example 22 (pTC) expressing the CPRb gene, and Example 19 (pC) only using PCPRb are slightly higher than that of Control. In contrast, the titers of DDA of Example 11 (p18), Example 12 (pC18), Example 15 (p19) and Example 16 (pC19) are all significantly improved compared to that of Control.

The results indicate that the gene editing system of Candida viswanathii of the present disclosure and uses thereof can effectively increase the titer of DDA by using the endogenous promoter or PcYP52A13 to drive the expression of the CYP52A18 gene, the CYP52A19 gene or the CPRb gene.

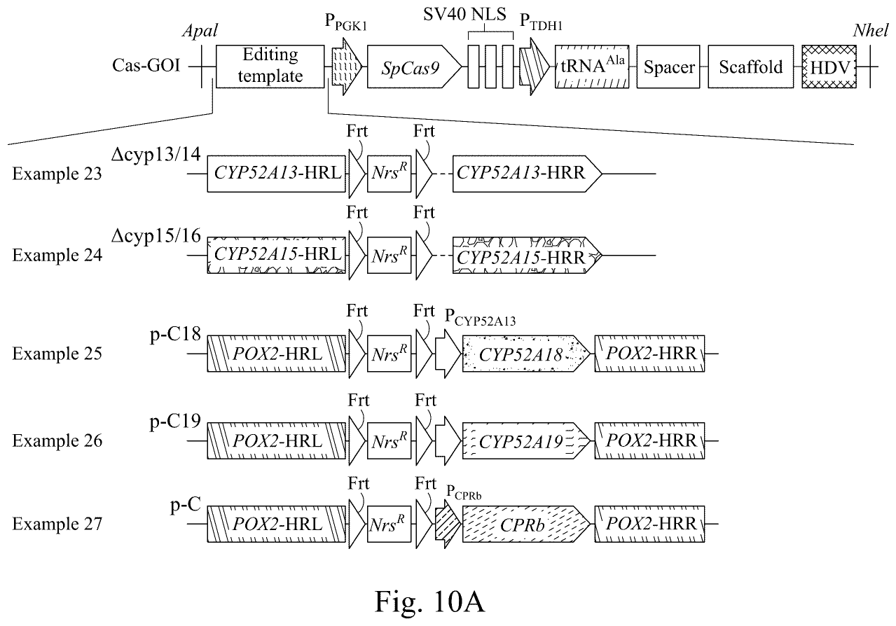

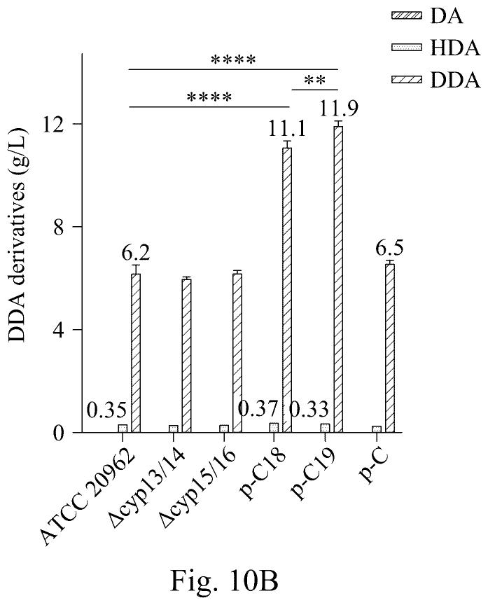

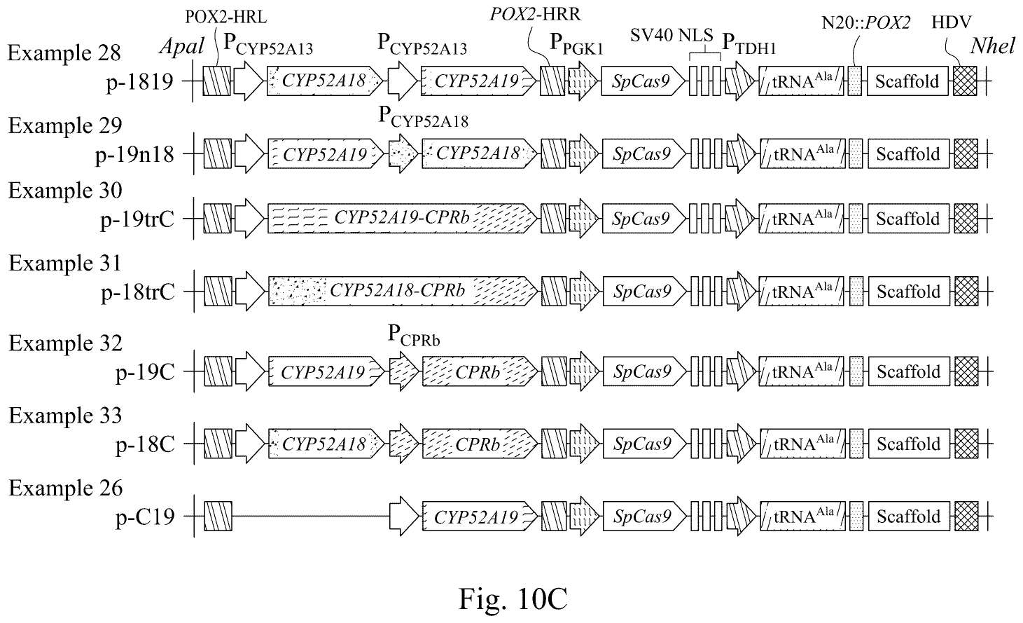

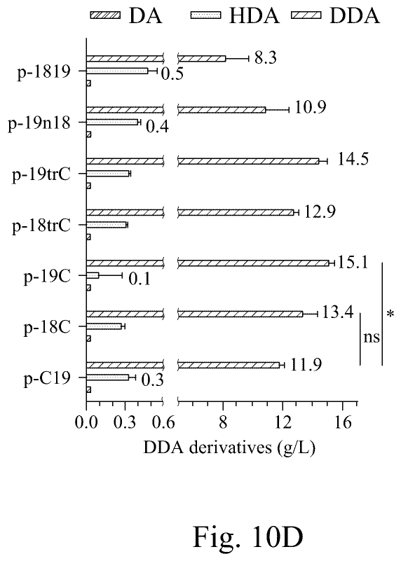

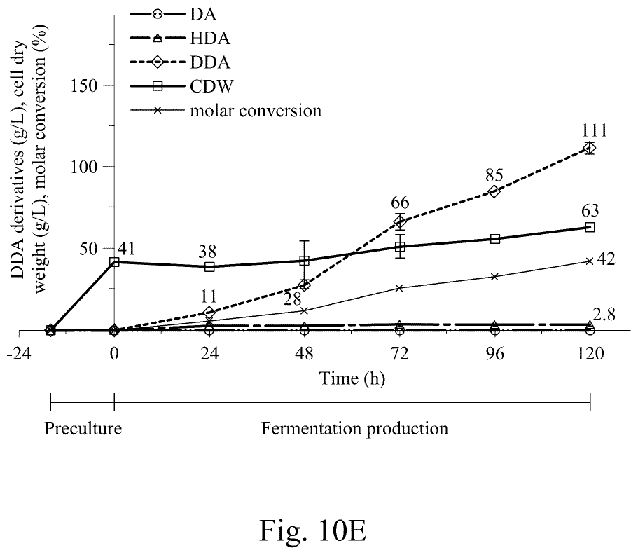

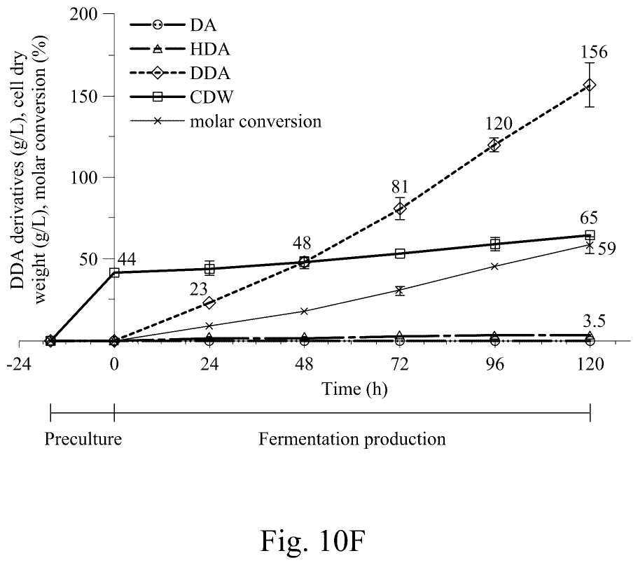

Reference is made to FIG. 10A and FIG. 10B. FIG. 10A is a schematic view showing constructions of Example 23 (Δcyp13/14), Example 24 (Δcyp15/16), Example 25 (p-C18), Example 26 (p-C19) and Example 27 (p-C) of the present disclosure for screening integrated site. FIG. 10B shows analytical results of titer of dodecanedioic acid from culture supernatants of Example 23 to Example 27 of the present disclosure cultured in shake flasks. In order to further improve the titer of DDA, a linear fragment (represented as Cas-GOI in FIG. 10A) including an editing template is experimentally constructed. Examples 23 to 27 are constructed based on Cas-GOI. The difference between Example 23 to Example 27 are the construction of the first homology arm, the second homology arm and the expression cassette represented by the editing template, wherein the first homology arm and the second homology arm correspond to different specific fragments of different genes on the chromosome of Candida viswanathii for deletion at the enzyme (CYP52A13/14 or CYP52A15/16) that may compete with the conversion of dodecane to DDA in Candida viswanathii. For example, the first homology arm of the sequence referenced as SEQ ID NO: 35 in Example 23 (Δcyp13/14) (represented as CYP52A13-HRL in FIG. 10A) and the second homology arm of the sequence referenced as SEQ ID NO: 36 (represented as CYP52A13-HRR in FIG. 10A), and the first homology arm of the sequence referenced as SEQ ID NO: 37 (represented as CYP52A15-HRL in FIG. 10A) and the second homology arm of the sequence referenced as SEQ ID NO: 38 (represented as CYP52A15-HRR in FIG. 10A) in Example 24 (Δcyp15/16). Alternatively, the expression cassette for the conversion of dodecane to DDA can be integrated together with deletion of the enzyme (PDX2) of the competing pathway. For example, the first homology arm of the sequence referenced as SEQ ID NO: 39 (represented as PDX2-HRL in FIG. 10A) and the second homology arm of the sequence referenced as SEQ ID NO: 40 (represented by PDX2-HRR in FIG. 10A) in Example 25 (p-C18), Example 26 (p-C19) and Example 27 (p-C). The spacer of Cas-GOI is the position of the targeting sequence corresponding to the target of the gene to be integrated in each Example. For example, N20::CYP52A13/14 of the sequence referenced as SEQ ID NO: 41 used in Example 23, N20::CYP52A15/16 of the sequence referenced as SEQ ID NO: 42 used in Example 24, N20::PDX2 of the sequence referenced as SEQ ID NO: 43 in Example 25 and Example 26. After electroporating Cas-GOI of Example 23 to Example 27 into Candida viswanathii respectively, Candida viswanathii containing Example 23 to Example 27 are cultured in shake flasks to produce DDA respectively, and then the titer of DDA in the culture supernatants is analyzed by GC-FID. After 24 hours of shake flask culture, the titers of DDA of Example 23 (Δcyp13/14), Example 24 (Δcyp15/16) and Example 27 (p-C) are all comparable to DDA produced by Candida viswanathii (Control), while titers of DDA of Example 25 (p-C18) and Example 26 (p-C19) are 90% higher than that of Control.

The results indicate again that the gene editing system of Candida viswanathii of the present disclosure and uses thereof can significantly improve increase the production of DDA by using PcYP52A13 to drive the expression of the CYP52A18 gene or the CYP52A19 gene and simultaneously blocking the enzyme (the PDX2 gene) in the ω-hydroxylation pathway.

Reference is made to FIG. 10C and FIG. 10D. FIG. 10C is a schematic view showing constructions of expression cassettes of Example 26 (p-C19), Example 28 (p-1819), Example 29 (p-19n18), Example 30 (p-19trC), Example 31 (p-18trC), Example 32 (p-19C) and Example 33 (p-18C) of the present disclosure for screening and optimizing enzymes for producing dodecanedioic acid. FIG. 10D shows analytical results of titer of dodecanedioic acid from culture supernatants of Example 26 and Example 28 to Example 33 of the present disclosure cultured in shake flasks. The results in the previous paragraph show that the selection of the exogenous gene promoter in the expression cassette and the selection of CYP or CPRb encoded by the exogenous gene significantly affect the titer of DDA. In order to further enhance the ability of the transformant to produce DDA, Example 28 to Example 33 are constructed. Example 26 and Example 28 to Example 33 are used to test DDA production performances together, so as to obtain a performance of the expression cassette for converting dodecane into DDA. Example 28 (p-1819), Example 29 (p-19n18), Example 32 (p-19C) and Example 33 (p-18C) express the CYP52A18 gene (represented as CYP52A18 in FIG. 100), the CYP52A19 gene (represented as CYP52A19 in FIG. 100) or the CPRb gene (represented as CPRb in FIG. 100) individually with two expression cassettes. Example 30 (p-19trC) and Example 31 (p-18trC) expresses the CYP52A18-CPRb genes with the sequence referenced as SEQ ID NO: 44 with a single expression cassette (represented as CYP52A18-CPRb in FIG. 100) or the CYP52A19-CPRb gene with the sequence referenced as SEQ ID NO: 45 (represented as CYP52A19-CPRb in FIG. 100). The results in FIG. 10D show that Example 28 (p-1819) and Example 29 (p-19n18) co-expressing the CYP52A18 gene and the CYP52A19 gene respectively give lower titer of DDA (8.3-10.9 g/L) than Example 26 (p-C19) expressing the CYP52A19 gene alone under P_{CYP52A13} (11.9 g/L). Conversely, the titers of DDA (12.9-15.1 g/L) of Example 32 (p-19C) and Example 33 (p-18C) co-expressing the CYP52A18 gene (or the CYP52A19 gene) and the CPRb gene respectively and Example 30 (p-19trC) and Example 31 (p-18trC) fusion co-expressing the CYP52A18-CPRb gene or the CYP52A19-CPRb gene are all greater than that of Example 26 (p-C19). Among these transformants, P-19C strain that transformed Example 32 (p-19C) can yield the highest DDA (15.1 g/L) within 24 hours.

Reference is made to FIG. 10E and FIG. 10F. FIG. 10E shows analytical results of titer of dodecanedioic acid by fermentation production of Candida viswanathii. FIG. 10F shows analytical results of titer of dodecanedioic acid by fermentation production of P-19C strain of the present disclosure. In order to evaluate the potential of the P-19C strain for large-scale production of DDA, the P-19C strain is inoculated and pre-cultured in a 3 L fermenter. After 16 hours, dodecane (3.6 g/h) and a feed solution (containing urea and glucose) are added in a fed-batch mode to induce DDA production. In addition, Candida viswanathii is cultured and induced in the same manner as Control. The results showed that the P-19C strain grows with similar rate and final cell dry weight (CDW) (65 g/L) but produces DDA at a faster rate (48 g/L) within 48 hours compared to Control. Thereafter, DDA production of P-19C strain increases nearly linear and reaches 156 g/L at 120 hours. The corresponding productivity (1.3 g/L/h) and molar conversion (59%) of P-19C strain are about 41% higher than those of Candida viswanathii.

### 3.2 Screening and Optimizing of Enzymes for Producing Dodecanedioic Acid and Establishing of a Transformant for Producing Dodecanedioic Acid

In Candida viswanathii, long-chain fatty alcohol oxidase 2 (FAO2) participates in the step of catalyzing the regeneration of long-chain alcohols into corresponding dicarboxylic acids through a series of oxidation reactions. The expression of FAO2 can be transiently induced by long-chain alkanes, but then sharply down-regulated, which affects the metabolic pathways that produce dicarboxylic acids. Therefore, a linear fragment co-expressing the FAO2 gene, the CYP52A19 gene and the CPRb gene is constructed to optimize the production of DDA. Since the linear fragment of Example 32 (p-19C) with the highest titer of DDA is very large (about 18.9 kb), integration of the FAO2 gene into Example 32 (p-19C) for co-integration of the CYP52A19 gene, the CPRb gene and the FAO2 gene is an extremely challenge. In order to solve the problem, the first gene editing fragment and the second gene editing fragment are designed experimentally, and different from the gene editing system of Candida viswanathii according to the aforementioned embodiment completing the recombination before transformation, the first gene editing fragment and the second gene editing fragment are recombined after being co-transformed into Candida viswanathii in this embodiment.

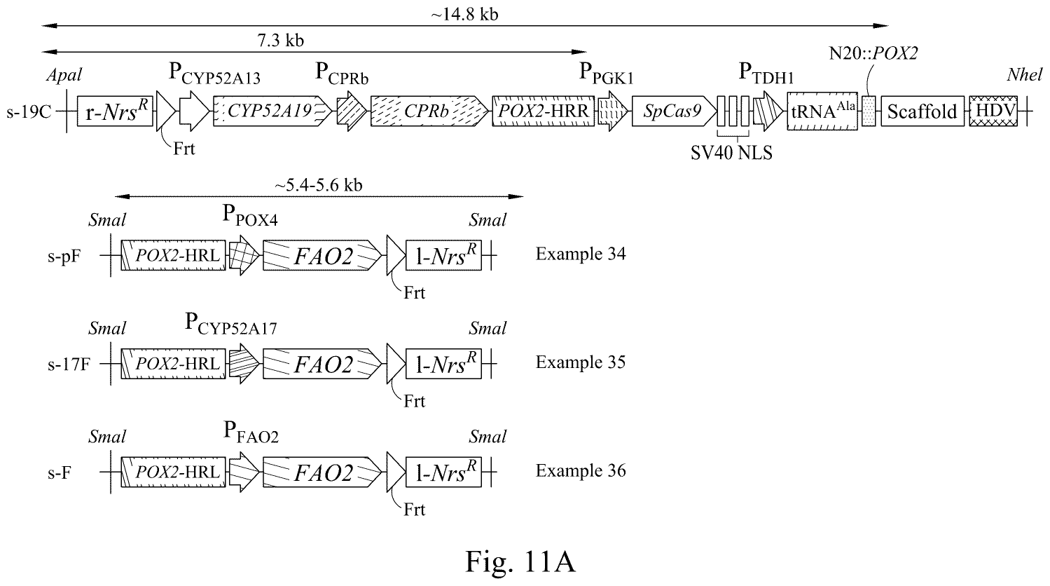

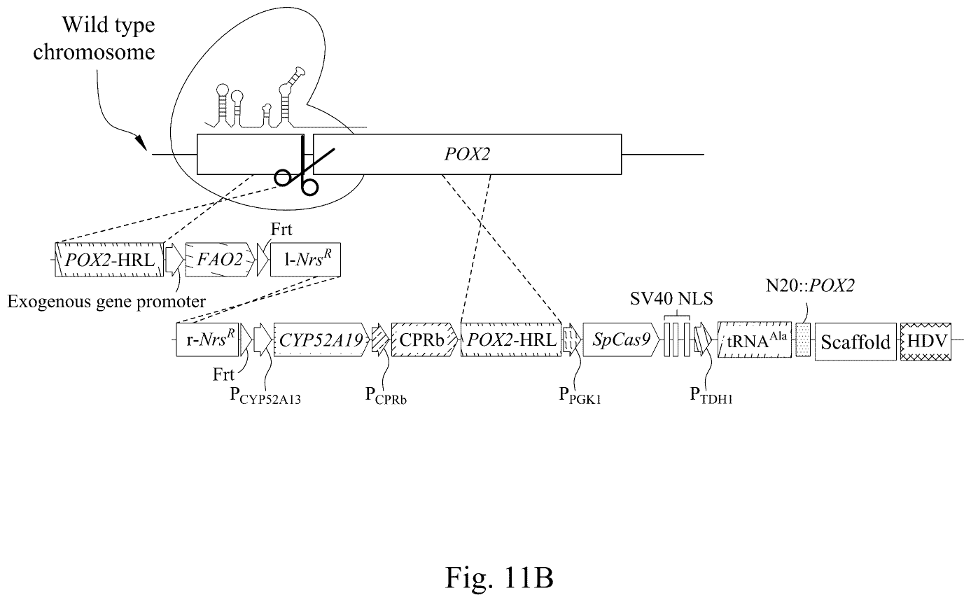

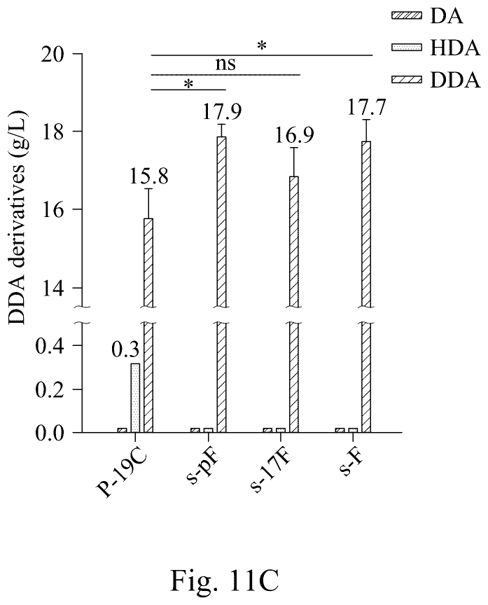

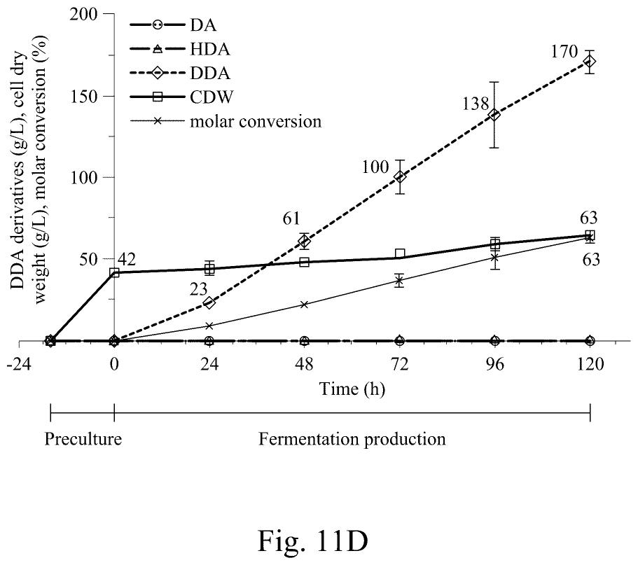

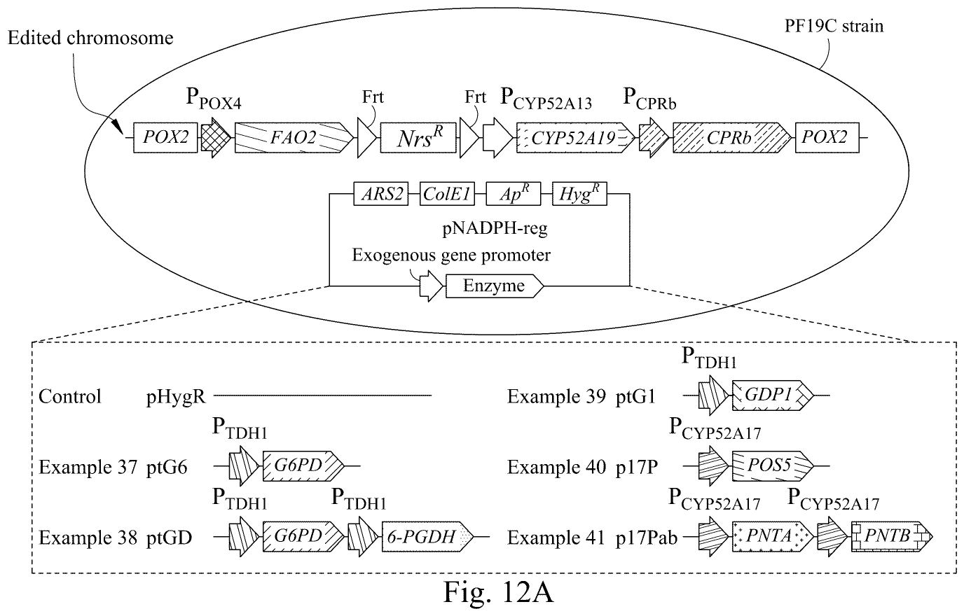

Reference is made to FIG. 11A and FIG. 11B. FIG. 11A is a schematic view showing constructions of expression cassettes of Example 34 (s-pF), Example 35 (s-17F) and Example 36 (s-F) of the present disclosure for screening enzymes of the long-chain alcohol oxidation pathway, and FIG. 11B is a schematic view showing the gene editing system of Candida viswanathii of the present disclosure constructing Example 34 to Example 36 in Candida viswanathii. Examples 34 to 36 are experimentally constructed as the first gene editing fragment, which successively included the first homology arm (represented as PDX2-HRL in FIG. 11A and FIG. 11B), an expression cassette for optimizing the long-chain alcohol oxidation pathway and a first screening gene fragment (represented as 1-Nrs^{R} in FIG. 11A and FIG. 11B). In the expression cassette for optimizing the long-chain alcohol oxidation pathway, three different exogenous gene promoters are used to drive the FAO2 gene of the sequence referenced as SEQ ID NO: 46 (represented as FAO2 in FIG. 11A and FIG. 11B), and the exogenous gene promoter is selected from P_{POX4} of the sequence referenced as SEQ ID NO: 47, PcYP52A17 of the sequence referenced as SEQ ID NO: 48 and P_{FAO2} of the sequence referenced as SEQ ID NO: 49. Example 34 (s-pF) expressing the FAO2 gene under P_{POX4}, Example 35 (s-17F) expressing the FAO2 gene under PcYP52A17 and Example 36 (s-F) expressing the FAO2 gene under P_{FAO2} are constructed. The second gene editing fragment (represented as s-19C in FIG. 11A) is similar to Example 32 (p-19C), but segment from the first homology arm (represented as PDX2-HRL in FIG. 11A and FIG. 11B) to the Nrs^{R} gene is replaced by a second screening gene fragment (represented as r-Nrs^{R} in FIG. 11A and FIG. 11B). The second screening gene fragment has a homologous fragment (not shown) that is homologous to part of the Nrs^{R} gene, and the homologous fragment is about 400 bp.

The first screening gene fragment and the second screening gene fragment share the homologous fragment that is homologous to part of the Nrs^{R} gene. Therefore, when the first gene editing fragment and the second first gene editing fragment are simultaneously electroporated into Candida viswanathii, the first gene editing fragment and the second gene editing fragment are recombined by the homologous fragment homologous to part of the Nrs^{R} gene. The CRISPR-Cas9 system can simultaneously integrate the expression cassettes expressing the CYP52A19 gene, the CPRb gene and the FAO2 gene into the PDX2 gene of Candida viswanathii, which can be used to overcome the problem that DNA size of the gene editing fragment to be integrated is too large.

Reference is made to FIG. 11C and FIG. 11D. FIG. 11C shows analytical results of titer of dodecanedioic acid from culture supernatants of Example 34 to Example 36 of the present disclosure cultured in shake flasks, and FIG. 11D shows analytical results of titer of dodecanedioic acid by fermentation production of PF19C strain of the present disclosure. After 24 hours of shake flask culture, compared with the titer of DDA (15.8 g/L) produced by P-19C strain only expressing the CYP52A19 gene and the CPRb gene, the three transformants that transformed Example 34 (s-pF), Example 35 (s-17F) or Example 36 (s-F) all can yield higher DDA (16.9-17.9 g/L) than P-19C strain. Especially titer of the DDA (17.9 g/L) of the transformant (PF19C strain) that transformed Example 34 (s-pF) increases by 13% more than the P-19C strain. In order to evaluate the potential of PF19C strain to produce DDA on a large scale, PF19C strain is cultured in the 3 L fermenter. Compared with P-19C strain, PF19C strain grows at similar rate, and produces 170 g/L DDA and barely detectable intermediate HDA at 120 hours of fermentation production. The molar conversion rate is slightly increased to 63%, and the productivity reaches about 1.42 g/L/h.

The aforementioned results show that the gene editing system of Candida viswanathii of the present disclosure and uses thereof for co-expression of the exogenous genes of the enzyme producing dodecanedioic acid (the CYP52A19 gene and the CPRb gene) combined with the exogenous genes of enzymes of the long-chain alcohol oxidation pathway (the FAO2 gene) can significantly enhance oxidation reaction steps that converts HDA to DDA. Therefore, the gene editing system of Candida viswanathii of the present disclosure and uses thereof can be easily applied to the screening of enzymes to establish a stable transformant for producing dodecanedioic acid. And further, large fragments can be efficiently integrated into the gene of Candida viswanathii by the CRISPR-Cas9 system of split editing fragments.

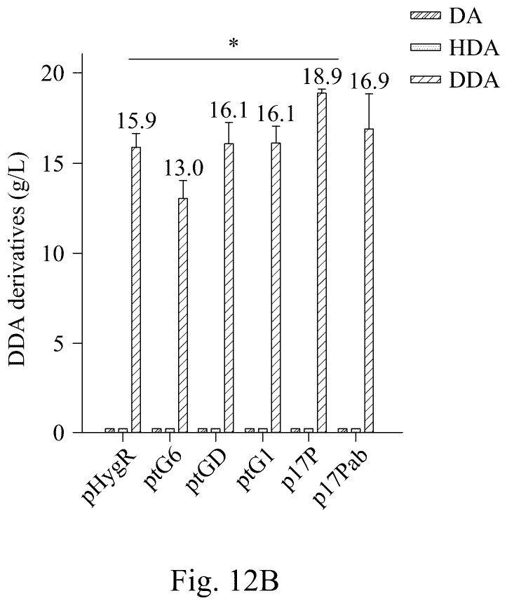

Reference is made to FIG. 12A and FIG. 12B. FIG. 12A is a schematic view showing constructions of expression cassettes of Example 37 (ptG6), Example 38 (ptGD), Example 39 (ptG1), Example 40 (p17P) and Example 41 (p17Pab) of the present disclosure for screening enzymes of NADPH-regenerating pathway. FIG. 12B shows analytical results of titer of dodecanedioic acid from culture supernatants of Example 37 to Example 41 of the present disclosure cultured in shake flasks. The transformants for producing dodecanedioic acid established in this test example are all based on the extension of the metabolic pathway catalyzed by CYP and CPRb. In the catalytic process, CYP and CPR irreversibly and continuously consume NADPH as a cofactor, and the reaction goes downstream in the metabolic pathway. Therefore, NADPH regeneration enhancement is necessary to establish a more stable and continuous transformant for producing dodecanedioic acid. In Candida viswanathii, NADPH can be regenerated by activating endogenous glucose 6-phosphate dehydrogenase (the G6DP gene of the sequence referenced as SEQ ID NO: 50, represented as G6DP in FIG. 12A), 6-phosphogluconate dehydrogenase (the 6-PGDH gene of the sequence referenced as SEQ ID NO: 51, represented as 6-PGDH in FIG. 12A) or NADH kinase (the POS5 gene of the sequence referenced as SEQ ID NO: 52, represented as POS5 in FIG. 12A); or by activating exogenous glyceraldehyde-3-phosphate dehydrogenase 1 (the GDP1 gene of the sequence referenced as SEQ ID NO: 53, represented as GDP1 in FIG. 12A) or transhydrogenase (the PNTA gene or the PNTB gene, in which the PNTA gene of the sequence referenced as SEQ ID NO: 54 and represented as PNTA in FIG. 12A, and the PNTB gene of the sequence referenced as SEQ ID NO: 55 and represented as PNTB in FIG. 12A). A plasmid template pNADPH-reg is experimentally constructed to express an exogenous gene promoter and one of the above-mentioned enzymes, and then transformed into PF19C strain as Example 37 to Example 41 to screen the expression cassette having better performance of the exogenous gene promoter and enzyme driving NADPH regeneration. PF19C strain expressing the Hyg^{R} gene (represented as Hyg^{R} in FIG. 12A) but not expressing the expression cassette is used as Control. After 24 hours of shake flask culture, compared with the titer of DDA (15.9 g/L) of Control, the titer of DDA (18.9 g/L) of Example 40 (p17P) expressing the POS5 gene (represented by POS5 in FIG. 12A) under PcYP52A17 is significantly higher than that of Control. The titer of DDA of other Examples is comparable with Control.

The aforementioned results indicate that the gene editing system of Candida viswanathii of the present disclosure and uses thereof to express the POS5 gene driven by PcYP52A17 can effectively catalyze the regeneration of NADPH and further improve the production of DDA.

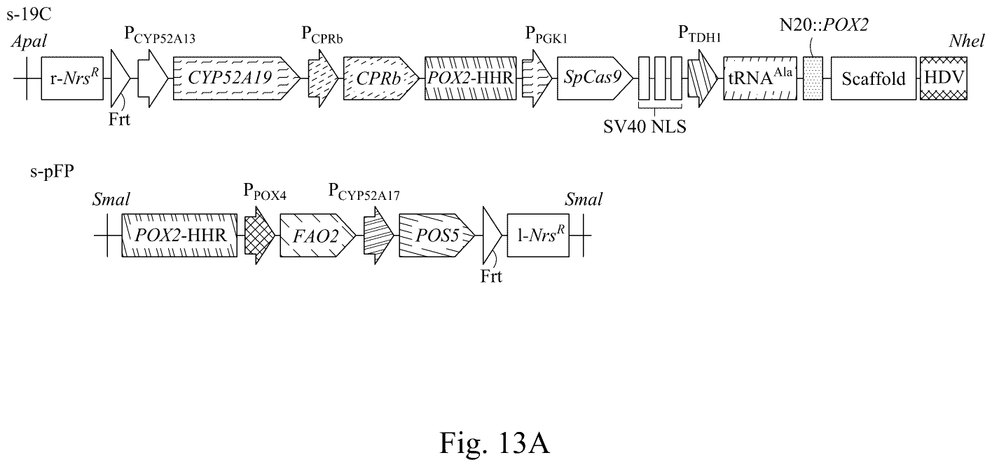

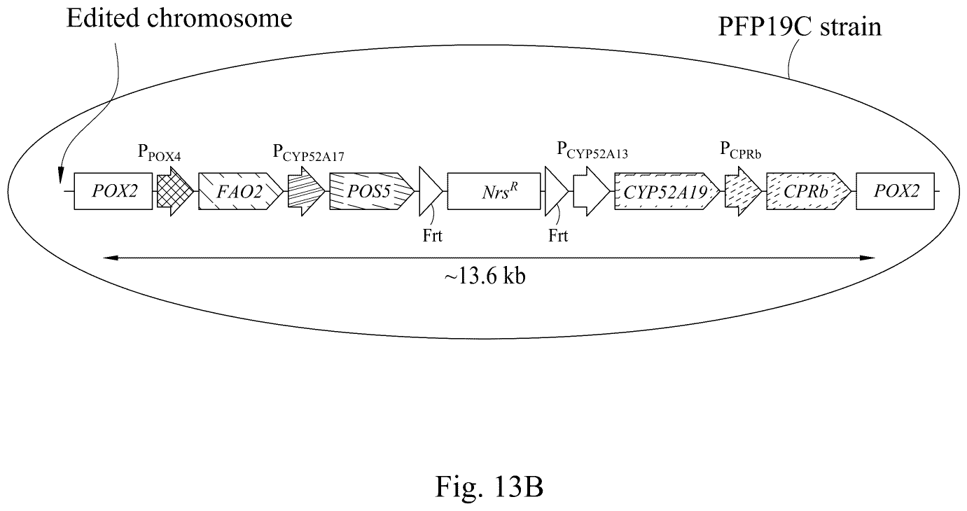

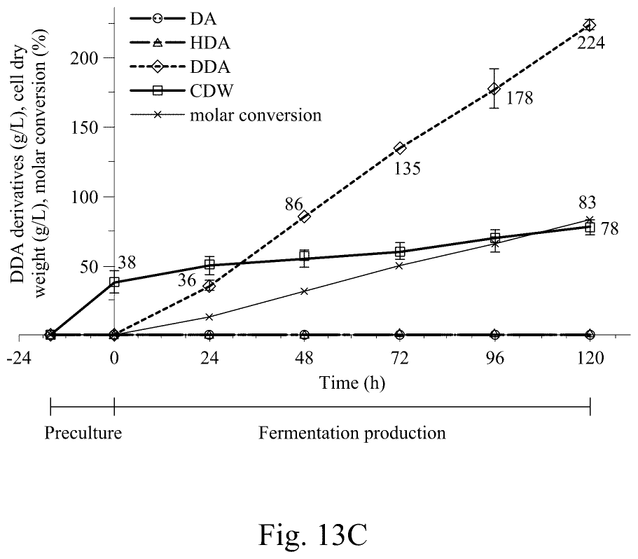

Reference is made to FIG. 13A to FIG. 13C. FIG. 13A is a schematic view showing construction of PFP19C strain of the present disclosure, FIG. 13B is a schematic view showing the edited chromosome of the PFP19C strain of the present disclosure, and FIG. 13C shows analytical results of titer of dodecanedioic acid by fermentation production of PFP19C strain of the present disclosure. In view of the above results, the CYP52A19 gene (represented as CYP52A19 in FIG. 13A and FIG. 13B), the CPRb gene (represented as CPRb in FIG. 13A and FIG. 13B), the FAO2 gene (represented as FAO2 in FIG. 13A and FIG. 13B) and the POS5 gene (represented as POS5 in FIG. 13A and FIG. 13B) are further co-integrated into the PDX2 gene (represented as PDX2 in FIG. 13B) of Candida viswanathii to construct a transformant for producing dodecanedioic acid (PFP19C strain). Then the established transformants are cultured in the 3 L fermenter to produce DDA. Unlike Candida viswanathii that has a lag phase and grows slowly after induction, PFP19C strain grows at 120 hours to reach 78 g/L dry cell weight (CDW) and produces DDA reaching 224 g/L at 120 hours with a nearly linear rate. Remarkably, the molar conversion concurrently increased to 83% with no detectable intermediate byproducts and the productivity reached 1.87 g/L/h. Overall, PFP19C accumulates the biomass needed for growth faster in the fermenter and produced about 102% more DDA than parent Candida viswanathii.

The above results indicate that the transformant for producing dodecanedioic acid constructed by the gene editing of Candida viswanathii of the present disclosure and use thereof can not only integrate a large gene editing fragment of about 13.6 kb into the genome of Candida viswanathii, the growth rate of the transformant for producing dodecanedioic acid can be significantly improved by co-expressing the POS5 gene, and the titer, the productivity, product purity and molar conversion of DDA produced by the oxidation reaction of dodecane can also be improved. Thus, the result verify the potential and feasibility of applying the gene editing system of Candida viswanathii of the present disclosure and the gene editing method thereof to construct the transformant for producing dodecanedioic acid.

In addition, please refer to Table 1, which is the comparison of titer of dodecanedioic acid by fermentation production between the transformant for producing dodecanedioic acid of the present disclosure established in the test examples and Candida viswanathii.

TABLE 1

| | | | | Molar | |
|---|---|---|---|---|---|
| | exogenous gene | Titer (g/L) | | conversion | Productivity |
| Strain | integrated | HDA | DDA | (%) | (g/L/h) |
| Candida | None | 2.8 | 111 | 42 | 0.93 |
| viswanathii | | | | | |
| P-19C | CYP52A19 gene, | 3.5 | 156 | 59 | 1.3 |
| | CPRb gene | | | | |
| PF19C | CYP52A19 gene, | 0 | 170 | 63 | 1.42 |
| | CPRb gene, | | | | |
| | FAO2 gene | | | | |
| PFP19C | CYP52A19 gene, | 0 | 224 | 83 | 1.87 |
| | CPRb gene, | | | | |
| | FAO2 gene, | | | | |
| | POS5 gene | | | | |

The results in Table 1 show that the gene editing system of Candida viswanathii of the present disclosure and the gene editing method thereof co-express the CYP52A18 gene or the CYP52A19 gene driven by the endogenous promoter or PcYP52A13 and the CPRb gene driven by PCPRb, which can significantly increase the production of DDA produced by Candida viswanathii. Furthermore, to further improve the titer, product purity, molar conversion rate and productivity of DDA, P-19C strain is constructed by integrating the above-mentioned exogenous gene into the PDX2 gene of Candida viswanathii to block the ω-hydroxylation pathway. Compared with Candida viswanathii, P-19C strain can increase the DDA production from 111 g/L to 156 g/L, and achieve a productivity of 1.3 g/L/h and a molar conversion of 59%. In addition, the gene editing system of Candida viswanathii of the present disclosure and the gene editing method thereof can be used in different optimization strategies to further construct the transformant for producing dodecanedioic acid, without being limited by the size of the gene editing target. For example, PF19C strain in which the FAO2 gene, the CYP52A19 gene, and the CPRb gene are co-integrated into the PDX2 gene of Candida viswanathii is constructed to strengthen the oxidation pathway for converting long-chain alcohols to dicarboxylic acids, and PFP19C strain in which the POS5 gene, the FAO2 gene, the CYP52A19 gene and the CPRb gene are co-integrated into the of the PDX2 gene of Candida viswanathii to further enhance NADPH regeneration. Thereby, the transformant for producing dodecanedioic acid of the present disclosure can finally effectively convert 200 g/L dodecane to 224 g/L DDA at a productivity of 1.87 g/L/h and a molar conversion rate of 83%, and there is almost no detectable by-product HDA. The above results indicate that the transformant for producing dodecanedioic acid and the method for producing dodecanedioic acid established by the gene editing system of Candida viswanathii of the present disclosure have high genome stability, high product selectivity and high substrate conversion rate. The results also verify the potential of the gene editing system of Candida viswanathii of the present disclosure and uses thereof in the field of the gene editing of Candida viswanathii and continuous production of DDA.

To sum up, the gene editing system of Candida viswanathii of the present disclosure and gene editing method thereof is the disclosure of firstly establishing the CRISPR-Cas9 system in Candida viswanathii. By screening the exogenous gene promoters, adjusting the combination of the first ribozyme and the second ribozyme and the recombination strategy of optimizing the homology arm can effectively overcome the operational difficulties and low recombination rate encountered in the conventional editing of Candida viswanathii with diploid. Thereby, the success rate and accuracy of gene editing of Candida viswanathii can be greatly increased to 60%, and gene editing methods such as deletion, mutation, replacement or fragment insertion can be easily and quickly realized without being affected by fragment size or insertion site, so as to obtain a gene-edited strain of Candida viswanathii that can be stably subcultured. Therefore, the gene editing system of Candida viswanathii of the present disclosure and the gene editing method thereof can be further applied to the construction of transformants for producing chemicals as a cell factory to participate in the sustainable economy of chemicals.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A gene editing system of Candida viswanathii, comprising:
a Candida viswanathii;
a first gene editing fragment, which successively comprises a first homology arm and a screening gene; and
a second gene editing fragment connected to a C-terminus of the first gene editing fragment, which successively comprises a second homology arm, a Cas9 expression cassette and a sgRNA cassette, wherein the Cas9 expression cassette successively comprises a Cas9 promoter, a Cas9 gene and three nuclear localization sequences, the sgRNA cassette successively comprises a sgRNA promoter, a first ribozyme, a targeting sequence, a scaffold and a second ribozyme, and the first gene editing fragment and the second gene editing fragment are constructed as a linear fragment for gene editing of a chromosome of the Candida viswanathii;
wherein the first homology arm and the second homology arm respectively correspond to a specific fragment of a gene on the chromosome of the Candida viswanathii, and the targeting sequence corresponds to a specific sequence of the gene on the chromosome of the Candida viswanathii.

2. The gene editing system of Candida viswanathii of claim 1, wherein the screening gene of the first gene editing fragment further comprises a first screening gene fragment, the second gene editing fragment further comprises a second screening gene fragment at an N-terminus of the second homology arm, the first screening gene fragment has a homologous fragment, the second screening gene fragment has the homologous fragment, and the first screening gene fragment and the second screening gene fragment are recombined into the linear fragment by the homologous fragment of the first screening gene fragment and the homologous fragment of the second screening gene fragment.

3. The gene editing system of Candida viswanathii of claim 2, wherein the first gene editing fragment and/or the second gene editing fragment further comprise at least one expression cassette, the at least one expression cassette comprises an exogenous gene promoter and an exogenous gene.

4. A gene editing method of Candida viswanathii, comprising:
constructing a first gene editing fragment, which successively comprises a first homology arm and a screening gene;
constructing a second gene editing fragment connected to a C-terminus of the first gene editing fragment, which successively comprises a second homology arm, a Cas9 expression cassette and a sgRNA cassette, wherein the Cas9 expression cassette successively comprises a Cas9 promoter, a Cas9 gene and three nuclear localization sequences, the sgRNA cassette successively comprises a sgRNA promoter, a first ribozyme, a targeting sequence, a scaffold and a second ribozyme, the first gene editing fragment and the second gene editing fragment are constructed as a linear fragment for gene editing of a chromosome of a Candida viswanathii, the first homology arm and the second homology arm respectively correspond to a specific fragment of a gene on the chromosome of the Candida viswanathii, and the targeting sequence corresponds to a specific sequence of the gene on the chromosome of the Candida viswanathii;
performing a transformation step, wherein the first gene editing fragment and the second gene editing fragment are transformed into the Candida viswanathii to obtain a transformant; and
performing a transformant cultivation step, wherein the transformant is cultivated in a selection medium at an editing temperature for an editing time, the Cas9 expression cassette expresses the Cas9 gene, the sgRNA cassette expresses the targeting sequence, the first homology arm and the second homology arm are respectively homologously recombined with the specific fragment, and the first gene editing fragment and the second gene editing fragment located between the first homology arm and the second homology arm are integrated into the gene of the transformant.

5. The gene editing method of Candida viswanathii of claim 4, wherein the screening gene of the first gene editing fragment further comprises a first screening gene fragment, the second gene editing fragment further comprises a second screening gene fragment at an N-terminus of the second homology arm, the first screening gene fragment has a homologous fragment, the second screening gene fragment has the homologous fragment, and the first screening gene fragment and the second screening gene fragment are recombined into the linear fragment by the homologous fragment of the first screening gene fragment and the homologous fragment of the second screening gene fragment.

6. The gene editing method of Candida viswanathii of claim 4, wherein the first gene editing fragment and/or the second gene editing fragment further comprise at least one expression cassette, the at least one expression cassette comprises an exogenous gene promoter and an exogenous gene.

7. A transformant for producing dodecanedioic acid, comprising:
a host cell, wherein the host cell is Candida viswanathii; and
at least two exogenous genes comprising CYP52A19 gene and CPRb gene, wherein the at least two exogenous genes are integrated into a chromosome of the host cell by the gene editing system of Candida viswanathii of claim 3.

8. The transformant for producing dodecanedioic acid of claim 7, wherein the at least two exogenous genes comprises CYP52A18 gene.

9. The transformant for producing dodecanedioic acid of claim 7, wherein the at least two exogenous genes are integrated into PDX2 gene of the chromosome of the host cell.

10. The transformant for producing dodecanedioic acid of claim 7, wherein the at least two exogenous genes comprises FAO2 gene.

11. The transformant for producing dodecanedioic acid of claim 7, wherein the at least two exogenous genes comprises POS5 gene.

12. A method for producing dodecanedioic acid, comprising:
providing a reaction substrate, wherein the reaction substrate comprises dodecane; and
performing a fermentation step, wherein the reaction substrate is inoculated with the transformant for producing dodecanedioic acid of claim 7, and then is cultured at a fermentation condition for a fermentation time to obtain a fermented substance, and the fermented substance comprises dodecanedioic acid.


SEQUENCE LISTING (55 sequences)

SEQ ID NO: 1 - DNA, 967 bp, synthetic construct
  source 1..967: mol_type = other DNA
gactaaaaggtatgtgttggtgtgaagaagaaagtggaagggaagctggtgatggtgggttcgtctatcccttttttatagttgcttgttagtagggtactctctagggactcgatgggggaaggttcttgatatttgcttagttcgagaaggttccagatgagcgagacatttttggtagcgacattgggcttgatcgatgatgatctgcacgacattttgtgttcttgcgacacgctgcactaccaagtgtaatctggctgaacggatcacaagataaacctctgaaaaattatctcagggcatgcaacaacaattatacatagaagagggagtcacgatatacacctgtgaaggaatcatgtggtcggctctccttgaactttgaattcatgcaattattaagaagaagcacaggtgagcaacccaccatacgttcatttgcaccacctgatgattaaaagccaaagaaagaaaaaaaaaaagaaacaggcggtgggaattgttacaacccacgcgaacccgaaaatggagcaatcttccccggggcctccaaataccaactcacccgagagagagaaagagacaccacccaccacgagacggagtatatccaccaaggtaagtaactcagggttaatgatacaggtgtacacagctccttccctagccattgagtgggtatcacatgacactggcaggttacaaccacgtttagtagttattttgtgcattccatggggatcaggaagtttggtttggtgggtgcgtctactgattcccctttgtctctgaaaatcttttccctagtggaacactttggctgaatgatataaattcaccttgattcccaccctcccttctttctctctctctctgttacacccaattgaattttcttttttttttttactttccctccttctttatcatcaagataagtaagtttatcaattgcctattcaga

SEQ ID NO: 2 - DNA, 4104 bp, synthetic construct
  source 1..4104: mol_type = other DNA
atggataaaaagtatagtattggtttagatattggtactaactctgtgggttgggcagttatcactgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgat

SEQ ID NO: 3 - DNA, 69 bp, synthetic construct
  source 1..69: mol_type = other DNA
  misc_feature 1..69: note = This sequence was repeated three times for the same SV40 NLS sequence
cctaagaagaaaagaaaagttgatccaaagaaaaagcgtaaggtggatcctaagaaaaagagaaaggtt

SEQ ID NO: 4 - DNA, 1455 bp, synthetic construct
  source 1..1455: mol_type = other DNA
aacgtggtatggttgtaagaaacatattgcaactggagatagcgatcgttcaatttattccgattttgtgggggaagtcgcccgctggtgggcgtgcgcgaatggcaaaagaaactcgaccatgcttttcatcatcccttaacagagcaatcatattttaaacgttcaagcaaaaagaaacgttggtttcggctaatgatcacctgaaaggcaaaatccttccatgtatgaacatgtaggttattccttttttttgcaacaccctcgggcagttgttcatattcccggaaaacaccaccactcggggctaagtggaagttctacaatcccggggaaataaggagccccggtgagcacgcgcacacaccaccttcacttcattttgtccgagggaagcagcacgtgaagtcggaacacgagaggagcatttcttctatttttttcttctctactgtgagtgcatgattatatatgtaatcaaaagcgatcaacttatggtagggtcgtgcacggcgcaccgggttccaaaatgatctgtgagggacaaaattcttttttttttccagcatgccgctggtggcaaataccgtggtggtatgatgcaccctatgccattgattcacaccaccaccattaatcaacaattgagagaggacaaaagtgaactattggtggtcgtcaggttatactcgtcagcttcggaatattacgtcccttcagtttgtgaaatgtcatcctggcgatgttcgagagagatcagtccgagagcgcgtggtaggagaaacggagcactgcagcaacaaaaaaaaaatccaaacccaggggggaggaagaagaacagccagggaaattgttcaccgacctgaccgtaaatttgctgctgaaagaaacgtgtcaaacaagaccaattggctcaattgaccctgagggagtactttgtctgccaccaatgcttccaccaaaacgctacttttgttttgcaatcggatggtgtgggtctggggtccacctgttttgttaagctacagaaggtggcatattcctctgatcaggtgctttttttcggctgctgctgctcgtggtggtgtagtggtagtggtgtgtgtgcgtgtgcgtgagggaggccgctttttgctctctgactcctcccaatcagaagttgctgtagcagtgaaacaacacaatggatgataatgccccgggcggtgcgtgtccgacacaaaccactacattttttagctgggagcatactgccactacgacccacccacccatggtcaacaaaaaaattctgacaaattataaaataacccttggattcccccttggaaaaatttttggtatttctctctttcttttccttttcctttccctcttctttttccctccatcaatcaattgacgttcagtaactcaattaattacatcacatccctcaattaaagaatttaaaca

SEQ ID NO: 5 - DNA, 522 bp, synthetic construct
  source 1..522: mol_type = other DNA
atgaaaatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctcttcacgtataaaactagacctcaagtctcgaacgaaacagcgatgtactggtactggttctcgggagcacaggatgacgcc

SEQ ID NO: 6 - DNA, 34 bp, synthetic construct
  source 1..34: mol_type = other DNA
gaagttcctattctctagaaagtataggaacttc

SEQ ID NO: 7 - DNA, 1083 bp, Candida viswanathi
  source 1..1083: mol_type = genomic DNA
cccacgatacacaaaagaatttgagacaatgaatcaatgacgcagaacaatttatttggccctctgttttatatctttttgtgaaggcaagttaaagccaacggtcgtgctgtatgggccgctaagatagatattcccactgcctgcattgttctgcttttctttcttttgctggagataggacctttgaggtcttgttgttttgcggctggaatgggtggggcaaagactgagccctggaagaaccattgtaaccgcactactattttgcaaaacctcgtgccgtctgaaaagcaataataacctacccctttttgcaatctttcattgtgcctaccttggcgtttgtgtcaatgagcactatatggactactccgctcaaccgtggacggtaacataccagcaccatcaactcatcgacggtcatttctcgtaacgaatgcctaatgccagtctattccatacaaggttttatcactgcgttgttttcttgcccatttcagaaatgggtcaaaggtcatcgccactagccacaaactcaatatgctaattataaatttccagatgggcactaaacaccacaatcctgaagccattaactctacgttgcaactcgtctcctcttttccccctctagactctagtctttgtccgattgagcttttcttttagacaagtttagttatctaaaaagcacaccattattatttttttagtatcctgcaagaagcgccggttttgttgaagagttaaactctatgtgtgctgtgcttgtcggtatgtgccgcaaccatgtgtcacgtggactgctcacgtggttcgtctttcgcactgccagagttaattggtggttattattttggcagcactcattcggttttttttttctgactcctcccctttaagatgggggaacaaaaaaaaagaatccaagattctgaaaaatcttatcggggctacatcgccatctcatcacacagatatagagacatagatacacgatggacagcaaaactataggtattttaggaggcggtcaattaggccgtatgattgttgaagctgctcatagactcaacatca

SEQ ID NO: 8 - DNA, 1001 bp, Candida viswanathi
  source 1..1001: mol_type = genomic DNA
gcttccgtttacttgtacggtaagacaaccagaccagatagaaaaatgggacatatcaacgtcactacgtcgtccatggatgacgccgaatcgagattgaagttcatcttgggcgagtctgaagacatcccggaatccttaaccgctcccaaagaagacccattggtgggtataataatgggttcggactctgacttgccagtgatggctgtcggtgccagaatcttgaagcagtttggtgtcccatttgagctcacgatcgtcagcgcccacagaaccccacacagaatgtcagaatacgccattgaggcaccaaagagaggccttaaatgtatcattgccggtgctggtggcgctgcccatttgccaggtatggttgccgccatgactccattgccagtcattggtgtccctgtgaagggatctactttggacggtgtcgattcattgcactccatcgtgcagatgccaagaggtatccccgtggctacggtggctatcaacaacagtaccaatgctgcgcttttggcaattagaatattgggcgcttacgactccaaatggttgacagaaatgaaccagtatatgttgaacatggagactgaagtgttgggcaaggctgaaacattagaggaaatcggctacgaagactacttgactgataagttaaagaagtagggtgtatatttctatttgaaggatgatgcaataataaaaaaaaactaataaatgataattgataaaaacgaattgaaacttggtgtaataaaaaacctctattatacaaaaacgctaaacttccctgtcccagttacctcaaaatttaagtgcctttctcttcttattcttaagttgtctttgtttatacgcctgtgatctcgaatctctccatccagaagtgctcaccttcttggtatctttaatttcgtcaacatccatggtcttagtctcatcctcattgtcttctttcttctgctgtttcttgagttcgtcctgcttct

SEQ ID NO: 9 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 10 - DNA, 43 bp, synthetic construct
  source 1..43: mol_type = other DNA
  misc_feature 1..6: note = Complementary to the first 6 nucleotides of the target sequence
nnnnnnctgatgagtccgtgaggacgaaacgagtaagctcgtc

SEQ ID NO: 11 - DNA, 68 bp, synthetic construct
  source 1..68: mol_type = other DNA
ggccggcatggtcccagcctcctcgctggcgccggctgggcaacatgcttcggcatggcgaatgggac

SEQ ID NO: 12 - DNA, 79 bp, Candida parapsilosis
  source 1..79: mol_type = other DNA
taaaatgggcgtgtggcgtagttggtagcgcgttcccttagcatgggaaaggtcatgagttcgattcttatctcgtcca

SEQ ID NO: 13 - DNA, 100 bp, Saccharomyces cerevisiae
  source 1..100: mol_type = other DNA
tttattttttgtcactattgttatgtaaaatgccacctctgacagtatggaacgcaaacttctgtctagtggataacagaatttttctatggccaattta

SEQ ID NO: 14 - DNA, 20 bp, synthetic construct
  source 1..20: mol_type = other DNA
tcatagactcaacatcaaga

SEQ ID NO: 15 - DNA, 20 bp, synthetic construct
  source 1..20: mol_type = other DNA
tacaagtaaacggaagcgtg

SEQ ID NO: 16 - DNA, 20 bp, synthetic construct
  source 1..20: mol_type = other DNA
gtgtaactagggagagtgcc

SEQ ID NO: 17 - DNA, 19 bp, synthetic construct
  source 1..19: mol_type = other DNA
gagggcaacttcaccagat

SEQ ID NO: 18 - DNA, 20 bp, synthetic construct
  source 1..20: mol_type = other DNA
gggcgcctaagaatatacgt

SEQ ID NO: 19 - DNA, 19 bp, synthetic construct
  source 1..19: mol_type = other DNA
aggcaaagtccgtgaagag

SEQ ID NO: 20 - DNA, 1143 bp, synthetic construct
  source 1..1143: mol_type = other DNA
atgaaaaagcctgaactcaccgcgacgtctgtcgagaagtttttgatcgaaaagttcgacagcgtctccgacttgatgcagctctcggagggcgaagaatctcgtgctttcagcttcgatgtaggagggcgtggatatgtcttgcgggtaaatagctgcgccgatggtttctacaaagatcgttatgtttatcggcactttgcatcggccgcgctcccgattccggaagtgcttgacattggggaattcagcgagagcttgacctattgcatctcccgccgtgcacagggtgtcacgttgcaagacttgcctgaaaccgaattgcccgctgttttgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggactacaccgtcagtgcgtccgtcgcgcaggctctcgatgagttgatgctttgggccgaggactgccccgaagtccggcacctcgtgcacgcggatttcggctccaacaatgtcttgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggaggcaaaggaattcgggagatgggggaggctaactgaaacacggaaggagacaataccggaaggaacccgcgctatgacggcaataaaaagacagaataaaacgcacgggtgttgggtcgtttgttcataa

SEQ ID NO: 21 - DNA, 1272 bp, synthetic construct
  source 1..1272: mol_type = other DNA
atgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgttgagtttcgatattgtcaataaatcactccagtttaaatacaagacgcaaaaagcaacaattttggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatttgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 22 - DNA, 695 bp, synthetic construct
  source 1..695: mol_type = other DNA
aaaaatccggggtagcgatgaggtagtgcaagttataccagagagcctcaatttgaacgtggtactgcacacaaaccgtagccctaaccctaattaaatgcacgtgacccacacaattttcaaccaccaacaacacaccaaaaatgtatgtacactcggagtggaaaatatttccccaggcataattgtggttgccaattatagtggagtgtttgttattagtataggagtactccttttatgtggaaaaatcgaaaaacataaaacagtgcagctatacactaacaaatggtatacccatggtcagacaacaccaaacaacacacccgtaatggttggtttggctaaatagagcatctcattttgtgtattattttatgttttggatatttttagcttgaaatttatataaaaatattttactccaattttcttgccaaattttgtacaaaaagtaaaaaatagaacttccaatttttgttaaacaaacttcaatctaacaaccgggatagtgtttggggggctgtgtaacaccactggtgtaataaaagtgccaatatttggaaattaattttttggtggtggacgtttcttcctaaaacacctagatgtgctagtataattgcacaaccagcacgcacactgcgtctggccggtcctggactacattttgtctctaacattcgtagctgcag

SEQ ID NO: 23 - DNA, 589 bp, synthetic construct
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 24 - DNA, 861 bp, synthetic construct
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 25 - DNA, 1569 bp, Candida viswanathi
  source 1..1569: mol_type = genomic DNA
atgactgtacacgatattatcgccacatacttcaccaaatggtacgtgatagtaccactcgctttgattgcttatagagtcctcgactacttctatggcagatacttgatgtacaagcttggtgctaaaccatttttccagaaacagacagacggctgtttcggattcaaagctccgcttgaattgttgaagaagaagagcgacggtaccctcatagacttcacactccagcgtatccacgatctcgatcgtcccgatatcccaactttcacattcccggtcttttccatcaaccttgtcaatacccttgagccggagaacatcaaggccatcttggccactcagttcaacgatttctccttgggtaccagacactcgcactttgctcctttgttgggtgatggtatctttacgttggatggcgccggctggaagcacagcagatctatgttgagaccacagtttgccagagaacagatttcccacgtcaagttgttggagccacacgttcaggtgttcttcaaacacgtcagaaaggcacagggcaagacttttgacatccaggaattgtttttcagattgaccgtcgactccgccaccgagtttttgtttggtgaatccgttgagtccttgagagatgaatctatcggcatgtccatcaatgcgcttgactttgacggcaaggctggctttgctgatgcttttaactattcgcagaattatttggcttcgagagcggttatgcaacaattgtactgggtgttgaacgggaaaaagtttaaggagtgcaacgctaaagtgcacaagtttgctgactactacgtcaacaaggctttggacttgacgcctgaacaattggaaaagcaggatggttatgtgtttttgtacgaattggtcaagcaaaccagagacaagcaagtgttgagagaccaattgttgaacatcatggttgctggtagagacaccaccgccggtttgttgtcgtttgttttctttgaattggccagaaacccagaagttaccaacaagttgagagaagaaattgaggacaagtttggactcggtgagaatgctagtgttgaagacatttcctttgagtcgttgaagtcctgtgaatacttgaaggctgttctcaacgaaaccttgagattgtacccatccgtgccacagaatttcagagttgccaccaagaacactaccctcccaagaggtggtggtaaggacgggttgtctcctgttttggtgagaaagggtcagaccgttatttacggtgtctacgcagcccacagaaacccagctgtttacggtaaggacgctcttgagtttagaccagagagatggtttgagccagagacaaagaagcttggctgggccttcctcccattcaacggtggtccaagaatctgtttgggacagcagtttgccttgacagaagcttcgtatgtcactgtcaggttgctccaggagtttgcacacttgtctatggacccagacaccgaatatccacctaagaaaatgtcgcatttgaccatgtcgcttttcgacggtgccaatattgagatgtattag

SEQ ID NO: 26 - DNA, 1623 bp, Candida viswanathi
  source 1..1623: mol_type = genomic DNA
atgtcgtcttctccatcgtttgcccaagaggttctcgctaccactagtccttacatcgagtactttcttgacaactacaccagatggtactacttcatacctttggtgcttctttcgttgaactttataagtttgctccacacaaggtacttggaacgcaggttccacgccaagccactcggtaactttgtcagggaccctacgtttggtatcgctactccgttgcttttgatctacttgaagtcgaaaggtacggtcatgaagtttgcttggggcctctggaacaacaagtacatcgtcagagacccaaagtacaagacaactgggctcaggattgttggcctcccattgattgaaaccatggacccagagaacatcaaggctgttttggctactcagttcaatgatttctctttgggaaccagacacgatttcttgtactccttgttgggtgacggtattttcaccttggacggtgctggctggaaacatagtagaactatgttgagaccacagtttgctagagaacaggtttctcacgtcaagttgttggagccacacgttcaggtgttcttcaagcacgttagaaagcaccgcggtcaaacgttcgacatccaagaattgttcttcaggttgaccgtcgactccgccaccgagttcttgtttggtgagtctgctgaatccttgagggacgaatctattggattgaccccaaccaccaaggatttcgatggcagaagagatttcgctgacgctttcaactattcgcagacttaccaggcctacagatttttgttgcaacaaatgtactggatcttgaatggctcggaattcagaaagtcgattgctgtcgtgcacaagtttgctgaccactatgtgcaaaaggctttggagttgaccgacgatgacttgcagaaacaagacggctatgtgttcttgtacgagttggctaagcaaaccagagacccaaaggtcttgagagaccagttattgaacattttggttgccggtagagacacgaccgccggtttgttgtcatttgttttctacgagttgtcaagaaaccctgaggtgtttgctaagttgagagaggaggtggaaaacagatttggactcggtgaagaagctcgtgttgaagagatctcgtttgagtccttgaagtcttgtgagtacttgaaggctgtcatcaatgaaaccttgagattgtacccatcggttccacacaactttagagttgctaccagaaacactaccctcccaagaggtggtggtgaagatggatactcgccaattgtcgtcaagaagggtcaagttgtcatgtacactgttattgctacccacagagacccaagtatctacggtgccgacgctgacgtcttcagaccagaaagatggtttgaaccagaaactagaaagttgggctgggcatacgttccattcaatggtggtccaagaatctgtttgggtcaacagtttgccttgaccgaagcttcatacgtcactgtcagattgctccaggagtttgcacacttgtctatggacccagacaccgaatatccaccaaaattgcagaacaccttgaccttgtcgctctttgatggtgctgatgttagaatgtactaa

SEQ ID NO: 27 - DNA, 1554 bp, Candida viswanathi
  source 1..1554: mol_type = genomic DNA
atgattgaacaaatcctagaatattggtatattgttgtgcctgtgttgtacatcatcaaacaactcattgcctacagcaagactcgcgtcttgatgaaacagttgggtgctgctccaatcacaaaccagttgtacgacaacgttttcggtatcgtcaacggatggaaggctctccagttcaagaaagagggcagagctcaagagtacaacgatcacaagtttgacagctccaagaacccaagcgtcggcacctatgtcagtattctttttggcaccaagattgtcgtgaccaaggatccagagaatatcaaagctattttggcaacccagtttggcgatttttctttgggcaagagacacgctctttttaaacctttgttaggtgatgggatcttcaccttggacggcgaaggctggaagcatagcagatccatgttaagaccacagtttgccagagaacaagttgctcatgtgacgtcgttggaaccacacttccagttgttgaagaagcatatccttaaacacaagggtgagtactttgatatccaggaattgttctttagatttactgtcgactcggccacggagttcttatttggtgagtccgtgcactccttaaaggacgaaactatcggtatcaaccaagacgatatagattttgctggtagaaaggactttgctgagtcgttcaacaaagcccaggagtatttgtctattagaattttggtgcagaccttctactggttgatcaacaacaaggagtttagagactgtaccaagctggtgcacaagtttaccaactactatgttcagaaagctttggatgctaccccagaggaacttgaaaagcaaggcgggtatgtgttcttgtatgagcttgtcaagcagacgagagaccccaaggtgttgcgtgaccagtctttgaacatcttgttggcaggaagagacaccactgctgggttgttgtcctttgctgtgtttgagttggccagaaacccacacatctgggccaagttgagagaggaaattgaacagcagtttggtcttggagaagactctcgtgttgaagagattacctttgagagcttgaagagatgtgagtacttgaaagcgttccttaacgaaaccttgcgtgtttacccaagtgtcccaagaaacttcagaatcgccaccaagaatacaacattgccaaggggtggtggtccagacggtacccagccaatcttgatccaaaagggagaaggtgtgtcgtatggtatcaactctacccacttagatcctgtctattatggccctgatgctgctgagttcagaccagagagatggtttgagccatcaaccagaaagctcggctgggcttacttgccattcaacggtgggccacgaatctgtttgggtcagcagtttgccttgaccgaagctggttacgttttggtcagattggtgcaagagttctcccacattaggctggacccagatgaagtgtatccaccaaagaggttgaccaacttgaccatgtgtttgcaggatggtgctattgtcaagtttgactag

SEQ ID NO: 28 - DNA, 1539 bp, Candida viswanathi
  source 1..1539: mol_type = genomic DNA
atgctcgatcagatcttacattactggtacattgtcttgccattgttggccattatcaaccagatcgtggctcatgtcaggaccaattatttgatgaagaaattgggtgctaagccattcacacacgtccaacgtgacgggtggttgggcttcaaattcggccgtgaattcctcaaagcaaaaagtgctgggagactggttgatttaatcatctcccgtttccacgataatgaggacactttctccagctatgcttttggcaaccatgtggtgttcaccagggaccccgagaatatcaaggcgcttttggcaacccagtttggtgatttttcattgggcagcagggtcaagttcttcaaaccattattggggtacggtatcttcacattggacgccgaaggctggaagcacagcagagccatgttgagaccacagtttgccagagaacaagttgctcatgtgacgtcgttggaaccacacttccagttgttgaagaagcatatccttaaacacaagggtgagtactttgatatccaggaattgttctttagatttactgtcgactcggccacggagttcttatttggtgagtccgtgcactccttaaaggacgaggaaattggctacgacacgaaagacatgtctgaagaaagacgcagatttgccgacgcgttcaacaagtcgcaagtctacgtggccaccagagttgctttacagaacttgtactggttggtcaacaacaaagagttcaaggagtgcaatgacattgtccacaagtttaccaactactatgttcagaaagccttggatgctaccccagaggaacttgaaaagcaaggcgggtatgtgttcttgtatgagcttgtcaagcagacgagagaccccaaggtgttgcgtgaccagtctttgaacatcttgttggcaggaagagacaccactgctgggttgttgtcctttgctgtgtttgagttggccagaaacccacacatctgggccaagttgagagaggaaattgaacagcagtttggtcttggagaagactctcgtgttgaagagattacctttgagagcttgaagagatgtgagtacttgaaggccgtgttgaacgaaactttgagattacacccaagtgtcccaagaaacgcaagatttgcgattaaagacacgactttaccaagaggcggtggccccaacggcaaggatcctatcttgatcaggaaggatgaggtggtgcagtactccatctcggcaactcagacaaatcctgcttattatggcgccgatgctgctgattttagaccggaaagatggtttgaaccatcaactagaaacttgggatgggctttcttgccattcaacggtggtccaagaatctgtttgggacaacagtttgctttgactgaagccggttacgttttggttagacttgttcaggagtttccaaacttgtcacaagaccccgaaaccaagtacccaccacctagattggcacacttgacgatgtgcttgtttgacggtgcacacgtcaagatgtcatag

SEQ ID NO: 29 - DNA, 2040 bp, Candida viswanathi
  source 1..2040: mol_type = genomic DNA
atggctttagacaagttagatttgtatgtcatcataacattggtggtcgctgtggccgcctattttgctaagaaccagttccttgatcagccccaggacaccgggttcctcaacacggacagcggaagcaactccagagacgtcttgctgacattgaagaagaataataaaaacacgttgttgttgtttgggtcccagaccggtacggcagaagattacgccaacaaattgtcaagagaattgcactccagatttggcttgaaaaccatggttgcagatttcgctgattacgattgggataacttcggagatatcaccgaagatatcttggtgtttttcatcgttgccacctacggtgagggtgaacctaccgacaatgccgacgagttccacacctggttgactgaagaagctgacactttgagtactttgagatataccgtgttcgggttgggtaactccacctacgagttcttcaatgctattggtagaaagtttgacagattgttgagtgagaaaggtggtgacagatttgctgaatatgctgaaggtgacgacggcactggcaccttggacgaagatttcatggcctggaaggataatgtctttgacgccttgaagaatgacttgaactttgaagaaaaggaattgaagtacgaaccaaacgtgaaattgactgagagagatgacttgtctgctgccgactcccaagtttccttgggtgagccaaacaagaagtacatcaactccgagggcatcgacttgaccaagggtccattcgaccacacccacccatacttggccaggatcaccgagaccagagagttgttcagctccaaggaaagacactgtattcacgttgaatttgacatttctgaatcgaacttgaaatacaccaccggtgaccatctagccatctggccatccaactccgacgaaaacatcaagcaatttgccaagtgtttcggattggaagataaactcgacactgttattgaattgaaggcattggactccacttacaccattccattcccaactccaattacttacggtgctgtcattagacaccatttagaaatctccggtccagtctcgagacaattctttttgtcgattgctgggtttgctcctgatgaagaaacaaagaagactttcaccagacttggtggtgacaaacaagaattcgccaccaaggttacccgcagaaagttcaacattgccgatgccttgttatattcctccaacaacactccatggtccgatgttccttttgagttccttattgaaaacatccaacacttgactccacgttactactccatttcttcttcgtcgttgagtgaaaaacaactcatcaatgttactgcagtcgttgaggccgaagaagaagccgatggcagaccagtcactggtgttgttaccaacttgttgaagaacattgaaattgcgcaaaacaagactggcgaaaagccacttgttcactacgatttgagcggcccaagaggcaagttcaacaagttcaagttgccagtgcacgtgagaagatccaactttaagttgccaaagaactccaccaccccagttatcttgattggtccaggtactggtgttgccccattgagaggtttcgttagagaaagagttcaacaagtcaagaatggtgtcaatgttggcaagactttgttgttttatggttgcagaaactccaacgaggactttttgtacaagcaagaatgggccgagtacgcttctgttttgggtgaaaactttgagatgttcaatgccttctctagacaagacccatccaagaaggtttacgtccaggataagattttagaaaacagccaacttgtgcacgaattgttgaccgaaggtgccattatctacgtctgtggtgacgccagtagaatggccagagacgtccagaccacgatctccaagattgttgccaaaagcagagaaatcagtgaagacaaggccgctgaattggtcaagtcctggaaagtccaaaatagataccaagaagatgtttggtag

SEQ ID NO: 30 - DNA, 1018 bp, Candida viswanathi
  source 1..1018: mol_type = genomic DNA
gttgcactcttgccataggcatgaaaataggccgttatagtactatatttaataagcgtaggagtataggatgcatatgaccggtttttctatatttttaagataatctctagtaaattttgtattctcagtaggatttcatcaaatttcgcaaccaattctggcgaaaaaatgattcttttacgtcaaaagctgaatagtgcagtttaaagcacctaaaatcacatatacagcctctagatacgacagagaagctctttatgatctgaagaagcattagaatagctactatgagccactattggtgtatatattagggattggtgcaattaagtacgtactaataaacagaagaaaatacttaaccaatttctggtgtatacttagtggtgagggaccttttctgaacattcgggtcaaacttttttttggagtgcgacatcgatttttcgtttgtgtaataatagtgaacctttgtgtaataaatcttcatgcaagacttgcataattcgagcttgggagttcacgccaatttgacctcgttcatgtgataaaagaaaagccaaaaggtaattagcagacgcaatgggaacatggagtggaaagcaatggaagcacgcccaggacggagtaatttagtccacactacatctgggggttttttttttgtgcgcaagtacacacctggactttagtttttgccccataaagttaacaatctaacctttggctctccaactctctccgcccccaaatattcgtttttacaccctcaagctagcgacagcacaacacccattagaggaatggggcaaagttaaacacttttggcttcaatgattcctattcgctactacattcttctcttgttttgtgctttgaattgcaccatgtgaaataaacgacaattatatataccttttcatccctcctcctatatctctttttgctacattttgttttttacgtttcttgcttttgcactctcccactcccacaaagaaaaaaaaactacact

SEQ ID NO: 31 - DNA, 739 bp, Candida viswanathi
  source 1..739: mol_type = genomic DNA
cccggacttgtttggggcagcagtacactttgattgaagcgagctatttgctagtcaggttggcgcagacctaccgggtaatcgatttgctgccagggtcggcgtacccaccaagaaagaagtcgttgatcaatatgagtgctgccgatggggtggttgtaaagtttcacaaggatctagatggatatgtaaggtgtgtaggaggagcggagataaattagatttgattttgtgtaaggtttagcacgtcaagctactccgcactttgtgtgtagggagcacatactccgtctgcgcctgtgccaagagacggcccaggggtagtgtgtggtggtggaagtgcatgtgacacaataccctggttctggccaattggggatttagtgtaggtaagctgcgacctgaaacactcctcaacgcttgagacactggtgggtagagatgcgggccaggaggctattcttgtcgtgctacccgtgcacggaaaatcgattgagggaagaacaaatttatccgtgaaatccacagagcggataaatttgtcacattgctgcgttgcccacccacagcattctcttttctctctctttgtcttactccgctcctgtttccttatccagaaatacacaccaactcatataaagatacgctagcccagctgtctttctttttcttcactttttttggtgtgttgcttttttggctgctactttctacaaccaccaccaccaccaccacc

SEQ ID NO: 32 - DNA, 558 bp, Candida viswanathi
  source 1..558: mol_type = genomic DNA
tgccacagctgtctggatgctggccgacacactttcaacggtctggatttggtctagaattcttgcacatgcacaacaaggaaactcttacaatgacaacacttatgatctaattccagctgatcttgctaatccttatcaacgtagttgtgatcattgtttgtctgaattatacacaccagtggaagaatatggtctaatttgcacgtcccactggcattgtgtgtttgtggggggggggggggtgcacacatttttagtgccattctttgttgattacccctcccccctatcattcattcccacaggattagttttttcctcactggaattcgctgtccacctgtcaaccccccccccccccccccactgccctaccctgccctgccctgcacgtcctgtgttttgtgctgtgtctttcccacgctataaaagccctggcgtccggccaaggtttttccacccagccaaaaaaacagtctaaaaaatttggttgatcctttttggttgcaaggttttccaccaccacttccaccacctcaactattcgaacaaaag

SEQ ID NO: 33 - DNA, 754 bp, Candida viswanathi
  source 1..754: mol_type = genomic DNA
tgggggggggatcaactgattagcggaagattggtgttgcctgtggggttcttttatttttcatatgatttctttgcgcgagtaacatgtgccaatctagtttatgattagcgtacctccacaattggcatcttggacgggcgtgttttgtcttaccccaagccttatttagttccacagtctcgacggtgtctcgccgatgtcttctcccacccctcgcaggaatcattcgaagttgttgggggatctcctccgcagtttatgttcatgtctttcccactttggttgtgattggggtagcgtagtgagttggtgattttcttttttcgcaggtgtctccgatatcgaagtttgatgaatataggagccagatcagcatggtatattgcctttgtagatagagatgttgaacaacaactagctgaattacacaccaccgctaaacgatgcgcacagggtgtcaccgccaactgacgttgggtggagttgttgttggcagggccatattgctaaacgaagagaagtagcacaaaacccaaggttaagaacaattaaaaaaattcatacgacaattccacagccatttacataatcaacagcgacaaatgagacagaaaaaactttcaacatttcaaagttccctttttcctattacttctttttttctttccttcctttcatttcctttccttctgcttttattactttaccagtcttttgcttgtttttgcaattcctcatcctcctcctcacc

SEQ ID NO: 34 - DNA, 638 bp, Candida viswanathi
  source 1..638: mol_type = genomic DNA
agatatttattcattatttagtttgcctatttatttctcattacccatcatcattcaacactatatataaagttacttcggatatcattgtaatcgtgcgtgtcgcaattggatgatttggaactgcgcttgaaacggattcatgcacgaagcggagataaaagattacgtaatttatctcctgagacaattttagccgtgttcacacgcccttctttgttctgagcgaaggataaataattagacttccacagctcattctaatttccgtcacgcgaatattgaaggggggtacatgtggccgctgaatgtgggggcagtaaacgcagtctctcctctcccaggaatagtgcaacggaggaaggataacggatagaaagcggaatgcgaggaaaattttgaacgcgcaagaaaagcaatatccgggctaccaggttttgagccagggaacacactcctatttctgctcaatgactgaacatagaaaaaacaccaagacgcaatgaaacgcacatggacatttagacctccccacatgtgatagtttgtcttaacagaaaagtataataagaacccatgccgtcccttttctttcgccgcttcaacttttttttttttatcttacacacatcacgacc

SEQ ID NO: 35 - DNA, 1003 bp, Candida viswanathi
  source 1..1003: mol_type = genomic DNA
gtccaggtgtgcacctgcgcacaaaaaaaaaaaagaatactagatgtgttgtgggctaaatggcactgtcatgggcgtgtttccattgctttctactccgtgttccccttggggggtctgccaattaccttttggctcttcttttatcacatgaatgaggataaattgagttgatatggtgcaacgttatgtggaaaccttcaactgtgcccttgcattgtagaaaccaacaccatcaccaccccgtgtaacacacttcactgcaatgaaagttaacggaaaaaataaaaaaaagtcacaatagtcaataattctggcgccccccattgttttgttggttcggagtgctgcggagtttttttattttttctgttggagtttctcatcttttgcattgtttgcaaaatgtttgaaattctggctgccatttattttttattacccctctaaataatatggattacgtaatcgactgtcgatcatttgtcgtgcaaacaggttgcaaaaaccacaatcacatgcttgcataacgtctaggctttacagattacataatcagagtagcagtcaaacaatcacatggagatcgaccttctatctaagacatcgtccggagttacaaaacaaaactccttttgactcaacaggatggaaggaaacaacaacctggaacgatcccatgtgaatgcaacacacacacacacacgcaccccctatcaataaataacaatccgtgaattaacctaatcaatagctacaaacagactaaaccaatcaaaaataacacatgatcctctaatacatctcaatgttggcaccgtcgaaaagggacatggtcaaatgcgacattttcctaggtggatattcggtgttggggtccatagacaagtgtccaaactcttggagcaatctgacagtgacatacgaagcttctgtcaaggcaaactgctgtcccaagcaaattcttggaccaccgttgaatggaaggaaggcccagccaagc

SEQ ID NO: 36 - DNA, 1048 bp, Candida viswanathi
  source 1..1048: mol_type = genomic DNA
tgttctccggctcaagggtgctgataaggttgatggaaaagattgggaatgtaaaagttgggatatctggacgattgagcgcttggatacgctcgagagtgaagtctatgagggtaccgtcactcttcttttttaacaattcaagtggagctttgaatccgaaataaccgtctgtttgtttctggaaaaacggtttagcaccaagcttgtacatcaagtatctgccgtaaaagtagtcgaggaccctataagcaatcaaagcgagtggtactatcacgtaccatttggtgatgtatgtggcgataatatcctgtgcagtcatggtcgtgaggtgtgtgtgtgttgatagacaaagaaaaagaaaaagttgaaacggcccaggaagagggaaggcatgggtccttattatactttttttgctttcgccagagaagctttcacatgtggggaggtctaaatgtccatgtgcgattcattgtgtcttggtggagttttttttttttgtctacgttaagtttttgtgcagaagaggagtgtgtcccctggctcaaaacctgatagcccggatattgctttccttgcgcattcaaaattttcctcgcattccacttgctatccgttatccttcccccgttgtactattcctgggggggggggggggggagagagagactgcgtttactggcaccccacattcatcggctacgtctaccccccccttccttattgttgcagttttcgcgtgacggatattttaatgaacttgtgaagtctaattatttatccttagctcagaagaagggcgtgtgaatgaggccaaaattgtcttaggagataatctcgtgttatctccgcatcgtgcatgaatccgttccaactacagtttcaaattacccaattgcaacacgcacgattacaatgatatatgagttccgaaataactttatatatattgttgaatgatgatgggtgatgagaaataaataggcaaactaaatagtgaataaatatctttaagcttctggacctgcagctctaacttcagcgg

SEQ ID NO: 37 - DNA, 971 bp, Candida viswanathi
  source 1..971: mol_type = genomic DNA
caaacaacgcgttgttgtcggtgactatcacatccaatcgctgcttcgaaataaagtacccaagtatgttcaagatgtagtttgctctcaaatagtaagacaatctggcactgtcggcaattgatgtgcccttcttgtaggatatgtcgccggcgtagtataacaactcggccgcgttcaattggtccaagtcaaactcgttcgacaataatgtggtaacttctaggaacgcttggttcaactccacttcgtctccattgggcaaggccacaggcttggatccatcgcctagtttctggcgcgattcttccagtggtgttggggttgtcaaaatgtggacaaggtcattcttgatgccgctgaaatctatggtgtctaaatcaatgtcttcctcaaacttaagggtgttgtagatatccccgaatgcctccagtgaccagttgaaagcgccgctcatttggttataatggttcacgttgggtaagtttgggcaatgttgtttacaaaaaattttctttttcgttttcaaacagtgctgatgagcgagcaacgaaggtaggggaagaaaaaaaaaaaaaaagtttgaacggttttggtcaaaacccactacagttattagtaccagaaaaatcaatcgccgattcaatttaaatacacaaactataaataacactagcaaggataagcaaccttagaacattctaacgtcagcaccatcaaagagtgacaaggtcaaggtgttctgcaattttggtgggtactcagcgtttggatccaggacaagtttccaaactcttggagcaatctgacagtgacgtatgaagcttcagtcaaggcaaactgctgacccaaacagattcttggaccaccattgaatggaacatatgcccagcccaactttctagtttctggctcgaaccatctttctggtctgaagacgtcggcgtcggcaccgtagatacttgggtctc

SEQ ID NO: 38 - DNA, 1039 bp, Candida viswanathi
  source 1..1039: mol_type = genomic DNA
cgtaggatccaacacgacgttaccgagcggcttggcgtggaacctgcgttccaagtactttgtgtggagcaagctgatgaagttcaacgaaagaagcaccaaagggatgaagtagtaccatctggtgtagttgtcaagaaagtactcgatgtaaggactagtggtagcgagaacctcctgagcaaacgatggagaagacgacatagtgtaggtttttttatttttcttctttgtgtgttgctgcactgattgtgggagtgggagagagtgcaaaagcaaaaagcaaaaagcaaaatggcagcaaaaagagacattggaggacagacgaaaaggtatatataattgtcgtttatttcacatggtgcaaatcaaaacacaaaacaagagagtagcaaataggaatcattgaaaccaaaagtgtttaactttgcccattcccctcatgggtgttgtgttgtcgttagctcgagggtgtaaaacgagcatctggaggcggagacactgggagagccaaaggtgagattgttaactttatggggcaataactaaagtccaggtgtgcacctgcgcacaaaaaaaaaaaagaatactagatgtgttgtgggctaaatggcactgtcatgggcgtgtttccattgctttctactccgtgttccccttggggggtctgccaattaccttttggctcttcttttatcacatgaatgaggataaattgagttgatatggtgcaacgttatgtggaaaccttcaactgtgcccttgcattgtagaaaccaacaccatcaccaccccgtgtaacacacttcactgcaatgaaagttaacggaaaaaataaaaaaaagtcacaatagtcaataattctggcgccccccattgttttgttggttcggagtgctgcggagtttttttattttttctgttggagtttctcatcttttgcattgtttgcaaaatgtttgaaattctggctgccatttattttttattacccctctaaataatatggattacgtaatcgactgtcgatcatttgtcg

SEQ ID NO: 39 - DNA, 960 bp, Candida viswanathi
  source 1..960: mol_type = genomic DNA
tgaatccacaatccccacctccaccgcggtggaaaaacctggaaaaaacaaaccaccatcaacaaccacccacctccactccactccccaaacaacaaccacacacacacctcatggcacgtcaaggactaatcagcatcctctccctcgtgtacttcatctacggtctcacgaaatgcgtcacgcagccgctcccaccacacctcatccaaggcggccacttccaattcctcaccaacgttctgcttgtcctcaccattctctacgtgctccagacgatcgtcacgccgacgagccggcacctccggacgcaacacaacgtcgtcgcgagtttggagttcaccgtcacggtgacctactggacgttgttcttgttggtgccgtcgtggctaaaccccgcggacgagtggggccgcgactgggtgcttgacgtcgcgatccacctctggccgttcgtctacttggtcgtgttcgacaaccaggacagaatccccccgaagagggcctggggtttgactggggccgtgattgttgtgtactggtgctaccttgaaagagtcgtttctgtgaatgccggggatggggtcacgaggtttgcgtacccttttttgaatgataggacgttgacgcagcggtttgggtggatggtgttgatttgggttgtgagttgtttgaactattacgttattggattgagaaatatgctatagatatgtggatgataaatacttgtttattcacagaaataatataaactttaactagagacaaaattcaatggggaggctctacttggataagatggcagcggctgctttagctctctcggatctttctctgttctcgagtgcagaacggttcaacatggcttccaaggcttcagagtatggtgccttgtagttgaatgggtcgttgacgtccttgacggattccaagtagttggtgtagatgttaccgtcg

SEQ ID NO: 40 - DNA, 940 bp, Candida viswanathi
  source 1..940: mol_type = genomic DNA
cgtcggtcttttcgtcaaaagtagcagtggtttcacaaccagcaacgttggaaccgtgacccaattcggtcataccgaaacaaccataaagctgcttgacaataccggcttccttcttgttggaccaaaagtcgaattgcttttgggtaccgttacctctaacacagttcaagaacaacccaatgttgaccaacattctgatacccaaggaagggtcaatgacagtcatcaactgcaatcttcttctaaacttaccgtaaggttcggtttccaagaatttggacatctggttgattctgttagcaaccaactctctttcttgcttttgggtgtagtcaaactgttctggtctggtttgcaagattgggtcacgttcaatggactggtacaagcgcaagatttgttcagactttccctcgtcaccttccaagaactcgtgcatttctttcaagtcccagtcggtgcgtgctctttcttcagcaacgtccttggctgggtgtggtgggtcctgggaggaaatgacatcagcaacttgctttggagttgtgtctgggtccttcttttgttggtggtcgtgaccgtcgtgtgattgactgagcatagccattgtgttaaagtagtattcagttttattaaagaggtgctaattaactgtgaatggttaatcaaaggtaaaactatacttgaagaagaggtaaaacaagggggttttagaaaaaaatcattcggtaaatcaagctggttttaaataattgacgggaatgaaccaaagtgtaagcccccccccaccgccaccaccaccttcaccactacgaacactatgatttttttttattaagcgtgcatgccaaaaatcaaatgtttttagtatcctccattttttctttctctctccctttgtttccttcgtccgctccccacctttcggcatcaccatcgg

SEQ ID NO: 41 - DNA, 20 bp, synthetic construct
  source 1..20: mol_type = other DNA
ccttgagccggagaacatca

SEQ ID NO: 42 - DNA, 20 bp, synthetic construct
  source 1..20: mol_type = other DNA
ggagtagcgataccaaacgt

SEQ ID NO: 43 - DNA, 20 bp, synthetic construct
  source 1..20: mol_type = other DNA
tcgttgattggtaagtacga

SEQ ID NO: 44 - DNA, 3561 bp, synthetic construct
  source 1..3561: mol_type = other DNA
atgattgaacaaatcctagaatattggtatattgttgtgcctgtgttgtacatcatcaaacaactcattgcctacagcaagactcgcgtcttgatgaaacagttgggtgctgctccaatcacaaaccagttgtacgacaacgttttcggtatcgtcaacggatggaaggctctccagttcaagaaagagggcagagctcaagagtacaacgatcacaagtttgacagctccaagaacccaagcgtcggcacctatgtcagtattctttttggcaccaagattgtcgtgaccaaggatccagagaatatcaaagctattttggcaacccagtttggcgatttttctttgggcaagagacacgctctttttaaacctttgttaggtgatgggatcttcaccttggacggcgaaggctggaagcatagcagatccatgttaagaccacagtttgccagagaacaagttgctcatgtgacgtcgttggaaccacacttccagttgttgaagaagcatatccttaaacacaagggtgagtactttgatatccaggaattgttctttagatttactgtcgactcggccacggagttcttatttggtgagtccgtgcactccttaaaggacgaaactatcggtatcaaccaagacgatatagattttgctggtagaaaggactttgctgagtcgttcaacaaagcccaggagtatttgtctattagaattttggtgcagaccttctactggttgatcaacaacaaggagtttagagactgtaccaagctggtgcacaagtttaccaactactatgttcagaaagctttggatgctaccccagaggaacttgaaaagcaaggcgggtatgtgttcttgtatgagcttgtcaagcagacgagagaccccaaggtgttgcgtgaccagtctttgaacatcttgttggcaggaagagacaccactgctgggttgttgtcctttgctgtgtttgagttggccagaaacccacacatctgggccaagttgagagaggaaattgaacagcagtttggtcttggagaagactctcgtgttgaagagattacctttgagagcttgaagagatgtgagtacttgaaagcgttccttaacgaaaccttgcgtgtttacccaagtgtcccaagaaacttcagaatcgccaccaagaatacaacattgccaaggggtggtggtccagacggtacccagccaatcttgatccaaaagggagaaggtgtgtcgtatggtatcaactctacccacttagatcctgtctattatggccctgatgctgctgagttcagaccagagagatggtttgagccatcaaccagaaagctcggctgggcttacttgccattcaacggtgggccacgaatctgtttgggtcagcagtttgccttgaccgaagctggttacgttttggtcagattggtgcaagagttctcccacattaggctggacccagatgaagtgtatccaccaaagaggttgaccaacttgaccatgtgtttgcaggatggtgctattgtcaagtttgacggatccgctggctccgctgctggttctggcgaattcgctaagaaccagttccttgatcagccccaggacaccgggttcctcaacacggacagcggaagcaactccagagacgtcttgctgacattgaagaagaataataaaaacacgttgttgttgtttgggtcccagaccggtacggcagaagattacgccaacaaattgtcaagagaattgcactccagatttggcttgaaaaccatggttgcagatttcgctgattacgattgggataacttcggagatatcaccgaagatatcttggtgtttttcatcgttgccacctacggtgagggtgaacctaccgacaatgccgacgagttccacacctggttgactgaagaagctgacactttgagtactttgagatataccgtgttcgggttgggtaactccacctacgagttcttcaatgctattggtagaaagtttgacagattgttgagtgagaaaggtggtgacagatttgctgaatatgctgaaggtgacgacggcactggcaccttggacgaagatttcatggcctggaaggataatgtctttgacgccttgaagaatgacttgaactttgaagaaaaggaattgaagtacgaaccaaacgtgaaattgactgagagagatgacttgtctgctgccgactcccaagtttccttgggtgagccaaacaagaagtacatcaactccgagggcatcgacttgaccaagggtccattcgaccacacccacccatacttggccaggatcaccgagaccagagagttgttcagctccaaggaaagacactgtattcacgttgaatttgacatttctgaatcgaacttgaaatacaccaccggtgaccatctagccatctggccatccaactccgacgaaaacatcaagcaatttgccaagtgtttcggattggaagataaactcgacactgttattgaattgaaggcattggactccacttacaccattccattcccaactccaattacttacggtgctgtcattagacaccatttagaaatctccggtccagtctcgagacaattctttttgtcgattgctgggtttgctcctgatgaagaaacaaagaagactttcaccagacttggtggtgacaaacaagaattcgccaccaaggttacccgcagaaagttcaacattgccgatgccttgttatattcctccaacaacactccatggtccgatgttccttttgagttccttattgaaaacatccaacacttgactccacgttactactccatttcttcttcgtcgttgagtgaaaaacaactcatcaatgttactgcagtcgttgaggccgaagaagaagccgatggcagaccagtcactggtgttgttaccaacttgttgaagaacattgaaattgcgcaaaacaagactggcgaaaagccacttgttcactacgatttgagcggcccaagaggcaagttcaacaagttcaagttgccagtgcacgtgagaagatccaactttaagttgccaaagaactccaccaccccagttatcttgattggtccaggtactggtgttgccccattgagaggtttcgttagagaaagagttcaacaagtcaagaatggtgtcaatgttggcaagactttgttgttttatggttgcagaaactccaacgaggactttttgtacaagcaagaatgggccgagtacgcttctgttttgggtgaaaactttgagatgttcaatgccttctctagacaagacccatccaagaaggtttacgtccaggataagattttagaaaacagccaacttgtgcacgaattgttgaccgaaggtgccattatctacgtctgtggtgacgccagtagaatggccagagacgtccagaccacgatctccaagattgttgccaaaagcagagaaatcagtgaagacaaggccgctgaattggtcaagtcctggaaagtccaaaatagataccaagaagatgtttggtag

SEQ ID NO: 45 - DNA, 3546 bp, synthetic construct
  source 1..3546: mol_type = other DNA
atgctcgatcagatcttacattactggtacattgtcttgccattgttggccattatcaaccagatcgtggctcatgtcaggaccaattatttgatgaagaaattgggtgctaagccattcacacacgtccaacgtgacgggtggttgggcttcaaattcggccgtgaattcctcaaagcaaaaagtgctgggagactggttgatttaatcatctcccgtttccacgataatgaggacactttctccagctatgcttttggcaaccatgtggtgttcaccagggaccccgagaatatcaaggcgcttttggcaacccagtttggtgatttttcattgggcagcagggtcaagttcttcaaaccattattggggtacggtatcttcacattggacgccgaaggctggaagcacagcagagccatgttgagaccacagtttgccagagaacaagttgctcatgtgacgtcgttggaaccacacttccagttgttgaagaagcatatccttaaacacaagggtgagtactttgatatccaggaattgttctttagatttactgtcgactcggccacggagttcttatttggtgagtccgtgcactccttaaaggacgaggaaattggctacgacacgaaagacatgtctgaagaaagacgcagatttgccgacgcgttcaacaagtcgcaagtctacgtggccaccagagttgctttacagaacttgtactggttggtcaacaacaaagagttcaaggagtgcaatgacattgtccacaagtttaccaactactatgttcagaaagccttggatgctaccccagaggaacttgaaaagcaaggcgggtatgtgttcttgtatgagcttgtcaagcagacgagagaccccaaggtgttgcgtgaccagtctttgaacatcttgttggcaggaagagacaccactgctgggttgttgtcctttgctgtgtttgagttggccagaaacccacacatctgggccaagttgagagaggaaattgaacagcagtttggtcttggagaagactctcgtgttgaagagattacctttgagagcttgaagagatgtgagtacttgaaggccgtgttgaacgaaactttgagattacacccaagtgtcccaagaaacgcaagatttgcgattaaagacacgactttaccaagaggcggtggccccaacggcaaggatcctatcttgatcaggaaggatgaggtggtgcagtactccatctcggcaactcagacaaatcctgcttattatggcgccgatgctgctgattttagaccggaaagatggtttgaaccatcaactagaaacttgggatgggctttcttgccattcaacggtggtccaagaatctgtttgggacaacagtttgctttgactgaagccggttacgttttggttagacttgttcaggagtttccaaacttgtcacaagaccccgaaaccaagtacccaccacctagattggcacacttgacgatgtgcttgtttgacggtgcacacgtcaagatgtcaggatccgctggctccgctgctggttctggcgaattcgctaagaaccagttccttgatcagccccaggacaccgggttcctcaacacggacagcggaagcaactccagagacgtcttgctgacattgaagaagaataataaaaacacgttgttgttgtttgggtcccagaccggtacggcagaagattacgccaacaaattgtcaagagaattgcactccagatttggcttgaaaaccatggttgcagatttcgctgattacgattgggataacttcggagatatcaccgaagatatcttggtgtttttcatcgttgccacctacggtgagggtgaacctaccgacaatgccgacgagttccacacctggttgactgaagaagctgacactttgagtactttgagatataccgtgttcgggttgggtaactccacctacgagttcttcaatgctattggtagaaagtttgacagattgttgagtgagaaaggtggtgacagatttgctgaatatgctgaaggtgacgacggcactggcaccttggacgaagatttcatggcctggaaggataatgtctttgacgccttgaagaatgacttgaactttgaagaaaaggaattgaagtacgaaccaaacgtgaaattgactgagagagatgacttgtctgctgccgactcccaagtttccttgggtgagccaaacaagaagtacatcaactccgagggcatcgacttgaccaagggtccattcgaccacacccacccatacttggccaggatcaccgagaccagagagttgttcagctccaaggaaagacactgtattcacgttgaatttgacatttctgaatcgaacttgaaatacaccaccggtgaccatctagccatctggccatccaactccgacgaaaacatcaagcaatttgccaagtgtttcggattggaagataaactcgacactgttattgaattgaaggcattggactccacttacaccattccattcccaactccaattacttacggtgctgtcattagacaccatttagaaatctccggtccagtctcgagacaattctttttgtcgattgctgggtttgctcctgatgaagaaacaaagaagactttcaccagacttggtggtgacaaacaagaattcgccaccaaggttacccgcagaaagttcaacattgccgatgccttgttatattcctccaacaacactccatggtccgatgttccttttgagttccttattgaaaacatccaacacttgactccacgttactactccatttcttcttcgtcgttgagtgaaaaacaactcatcaatgttactgcagtcgttgaggccgaagaagaagccgatggcagaccagtcactggtgttgttaccaacttgttgaagaacattgaaattgcgcaaaacaagactggcgaaaagccacttgttcactacgatttgagcggcccaagaggcaagttcaacaagttcaagttgccagtgcacgtgagaagatccaactttaagttgccaaagaactccaccaccccagttatcttgattggtccaggtactggtgttgccccattgagaggtttcgttagagaaagagttcaacaagtcaagaatggtgtcaatgttggcaagactttgttgttttatggttgcagaaactccaacgaggactttttgtacaagcaagaatgggccgagtacgcttctgttttgggtgaaaactttgagatgttcaatgccttctctagacaagacccatccaagaaggtttacgtccaggataagattttagaaaacagccaacttgtgcacgaattgttgaccgaaggtgccattatctacgtctgtggtgacgccagtagaatggccagagacgtccagaccacgatctccaagattgttgccaaaagcagagaaatcagtgaagacaaggccgctgaattggtcaagtcctggaaagtccaaaatagataccaagaagatgtttggtag

SEQ ID NO: 46 - DNA, 2115 bp, Candida viswanathi
  source 1..2115: mol_type = genomic DNA
atggctccatttttgcccgaccaggtcgactacaaacacgtcgacacccttatgttattatgtgacgggatcatccacgaaaccaccgtggacgaaatcaaagacgtcattgcccctgacttccccgccgacaaatacgaggagtacgtcaggacattcaccaaaccctccgaaaccccagggttcagggaaaccgtctacaacaccgtcaacgcaaacaccatggatgcaatccaccagttcattatcttgaccaatgttttgggatcaagggtcttggcaccagctttgaccaactcgttgactcctatcaaggacatgagcttggaagaccgtgaaaagttgttagcctcgtggcgtgactcccctattgctgctaaaaggaagttgttcaggttggtttctacgcttaccttggtcacgttcacgagattggccaatgagttgcatttgaaagccattcattatccaggaagagaagaccgtgaaaaggcttatgaaacccaggagattgacccttttaagtaccagtttttggaaaaaccgaagttttacggcgctgagttgtacttgccagatattgatgtgatcattattggatctggggccggtgctggtgtcgtggcccacactttgaccaacgacggcttcaagagtttggttttggaaaagggcagatactttagcaactccgagttgaactttgatgacaaggacggggttcaagaattataccaaagtggaggtactttgaccaccgtcaaccagcagttgtttgttcttgctggttccacttttggtggtggtaccactgtcaattggtcggcctgtcttaaaacgccattcaaggtgcgtaaggaatggtatgatgagtttggcgttgactttgctgccgatgaagcctacgacaaagcacaggattatgtttggcagcaaatgggagcttctaccgaaggcatcacccactctttggctaacgagattattattgaaggtggcaagaaattaggttacaaggccaaggtattagaccaaaacagcggtggtcatcctcatcacagatgcggtttctgttatttgggttgtaagcacggtatcaagcagggctctgttaataactggtttagagacgcagctgcccacggttctcagttcatgcaacaggttagagttttgcaaatccttaacaagaagggcatcgcttatggtatcttgtgtgaggatgttgtaaccggtgccaagttcaccattactggccccaaaaagtttgttgttgccgccggcgccttaaacactccatctgtgttggtcaactccggattcaagaacaagaacatcggtaagaacttaactttgcatccagtttctgtcgtgtttggtgattttggcaaagacgttcaagcagatcacttccacaactccatcatgactgctctttgttcagaagccgctgatttagacggcaagggtcatggatgcagaattgaaaccatcttgaacgctccattcatccaggcttcattcttaccatggagaggtagtaacgaggctagacgagacttgttgcgttacaacaacatggtggccatgttacttcttagtcgtgataccaccagtggttccgtttcgtcccatccaactaaacctgaagcattagttgtcgagtacgacgtgaacaagtttgacagaaactccatcttgcaggcattgttggtcactgctgacttgttgtacattcaaggtgccaagagaatccttagtccccaaccatgggtgccaatttttgaatccgacaagccaaaggataagagatcaatcaaggacgaggactatgtcgaatggagagccaaggttgccaagattccttttgacacctacggctcgccttatggttcggcgcatcaaatgtcttcttgtcgtatgtcaggtaagggtcctaaatacggtgctgttgataccgatggtagattgtttgaatgttcgaatgtttatgttgctgacgctagtcttttgccaactgctagcggtgctaatcctatggtcaccaccatgactcttgcaagacatgttgcgttaggtttggcagactccttgaagaccaaggccaagttgtag

SEQ ID NO: 47 - DNA, 516 bp, Candida viswanathi
  source 1..516: mol_type = genomic DNA
tatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttttttttctctgtgcttccccctcctgtgactttttccaccattgatataaaatcaactccatttccctaaaatctccccagattctaaaaacaacttcttctcttctgcttttcctttttttttgttatatttatttaccatcccttttttttgaatagttattccccactaacattgttcaaatcttcacgacata

SEQ ID NO: 48 - DNA, 581 bp, Candida viswanathi
  source 1..581: mol_type = genomic DNA
ttgtgtaggaggagcggagataaattagatttgattttgtgtaaggttttggatgtcaacctactccgcacttcatgcagtgtgtgtgacacaagggtgtactacgtgtgcgtgtgcgccaagagacagcccaagggggtggtagtgtgtgttggcggaagtgcatgtgacacaacgcgtgggttctggccaatggtggactaagtgcaggtaagcagcgacctgaaacattcctcaacgcttaagacactggtggtagagatgcggaccaggctattcttgtcgtgctacccggcgcatggaaaatcaactgcgggaagaataaatttatccgtagaatccacagagcggataaatttgcccacctccatcatcaaccacgccgccactaactacatcactcccctattttctctctctctctttgtcttactccgctcccgtttccttagccacagatacacacccactgcaaacagcagcaacaattataaagatacgccaggcccaccttctttctttttcttcacttttttgactgcaactttctacaatccaccacagccaccaccacagccgct

SEQ ID NO: 49 - DNA, 645 bp, Candida viswanathi
  source 1..645: mol_type = genomic DNA
gtcggcgaacttgacaaggtgttcaaggatgaaaagtttgccgtcaacgacaagattagattggttgagatcatgttagacactttcgatgctccagagaacttggttaagcaagctgagagatctgccaacaccaacaagtagagtttgtctatgttttccgtttgccttttctttctagtacgagacgttattgaacgaagtttttatatatctagatctaatacatattccatgtctgttcatttttgacggagtttcataaggtggcagtttctaatcaaaggtccgtcattggcgtcgtggcattggcggctcgcatcaactcgtatgtcaatattttctgttaactccgccagacatacgatcaaaacctacaagcaaaaaaattccacatgctttgtttgagatctccacaaacaacaacggggtaagaaaatcatggggcgattaatcatgccatctttgtaaatttctttgtttcaacatcaccctctttagtcaaaccttcacaggactgtctgctctactttgccacccagttcatatataaattaccaacttccaccgagcaccaccaacacctcaccccactctctcccccccccttttttttccagcttagacacacacttcaaactcgac

SEQ ID NO: 50 - DNA, 1500 bp, Candida viswanathi
  source 1..1500: mol_type = genomic DNA
atgtcttatgattcattcggtgactacgtcactatcgtcgttttcggtgcttccggtgacttggccagcaaaaaaaccttccctgccttgtttggcttgtttagagaaaagcaattgcccccaaccgtccagatcattggctatgccagatcccatttgtccgacaaggacttcaaaaccaagatctcctcccacttcaagggcggcgacgaaaaaaccaagcaagacttcttgaacttgtgtacttatatcagcgacccatacgacactgacgatggttacaagagattggaagccgccgctcaagaatacgaatccaagcacaacgtcaaggtccctgaaagattgttttacttggccttgcctccttctgtcttccacaccgtctgtgagcaagtcaagaagatcgtctaccctaaggacggtaagctcagaatcatcattgaaaagccgttcggacgtgatttggccacctaccgtgaattgcaaaagcaaatctccccattgttcaccgaagacgaactctacagaattgaccactacttgggtaaagaaatggtcaagaacttgttggttttgagattcggtaacgaattgttcagtgggatctggaacaacaagcacatcacctcggtgcaaatctccttcaaggaacccttcggtaccgaaggtagaggtggctactttgacaacattggtatcatcagagatgtcatgcaaaaccacttgttgcaagtcttgaccttgttgaccatggaaagaccagtctcttttgacccagaagctgtcagagacgaaaaggtcaaggttttgaaagcttttgacaagattgacgtcaacgacgttcttttgggacaatacgccaagtctgaggatggctccaagccaggttacttggatgactccaccgtcaagccaaactccaaggctgtcacctacgccgctttcagagtcaacatccacaacgaaagatgggacggtgttccaattgttttgagagccggtaaggctttagacgaaggtaaagttgaaattagaatccaattcaagccagttgccaaaggtatgtttaaggagatccaaagaaacgaattggttattagaatccaaccagacgaagccatctacttgaagatcaactccaagatcccaggtatctccaccgaaacttccttgaccgacttggacttgacttactccaagcgttactccaaggacttctggatcccagaagcatacgaagccttgatcagagactgttacttgggcaaccactccaactttgtcagagacgatgaattggaagttgcttggaagctcttcaccccattgttggaagccgttgaaaaagaagacgaagtcagcttgggaacctacccatacggatccaagggtcctaaagaattgagaaagtacttggtcgaccacggttacgtcttcaacgacccaggtacttaccaatggccattgaccaacaccgatgtcaagggtaagatctaa

SEQ ID NO: 51 - DNA, 2010 bp, Candida viswanathi
  source 1..2010: mol_type = genomic DNA
atgtcttcagctccaaagtatgtacctcaattcgtttttatcaattggaacaagagagataggacggcagaattccagagattggacaggaacaagaacaattaggcaatgggcgagcggaaatcaattggatctactcaattgatatttttaaccagaacccagccacttggcaatatactcaacatgtccgttccacccccctcctagaaattttcacattggctggaatttttttttaggaggagatgaaatcgttgagaaccgagcgttcctcgcccccaaattttttttctgttcggctcacacgcccaagggatcaaggataatcagctgcaaggaaaaaaaataaagtataaagagaatgccgtttttcccccacattgaaagttttttcagatttttgcaacccaacgtttatcaagttcttttttttttggcaacacgtcttcccaaatgtccaatcccaaagctgtctttaggttatctgctctatccaagcaaatcgcgaccaacaatactaacaatctgattgctagaggtgatatcggtttgattggtttagccgtcatgggtcaaaacttgatccttaacatggctgaccacggttacactgttgttgcttacaacagaaccacctccaaggttgaccacttcttggccaacgaagctaagggcaagtctatcctcggtgctcactccatccaagaattggttgaccaattgaagagaccaagaagaattatgcttttggtcaaggccggtgctcccgttgacagtttcattgaccaattggtcccatacttggaagaaggtgacatcatcattgacggtggtaactcccacttcccagacaccaacagaagatacgacgacttgaagaagaagggtatcttgtttgtcggttccggtgtctctggtggtgaagaaggtgccagaactggtccatctttgatgccaggtggtaacgaagctgcttggccacacatcaaggacatcttccaagacatctccgccaaggctgacggtgaaccatgttgtgactgggttggtccagcgggtgccggtcactacgtcaagatggtccacaacggtattgaatacggtgacatgcaattgatttgtgaagcctacgacctcatgaagagagttggtaagttctccaacaaggaaatgggtgacgttttcgccaagtggaacaagggtgttttggactctttcttgattgaaatcaccagagacattatgtactacgacgacccaactgacggtaccccattggttgaaaagatcttggacacagccggtcaaaagggtactggtaagtggactgctgtcaacgcccttgacttgggtatccctcttaccttggttggtgaagccgtcttctccagatgtttgtctgctttgaagaacgaaagagttgaagcctccaagcacttgccaggtccaaagattgatgaacttcctccaatcaccaacaaggaagaattcattgaccacttggaacaagccttgtacgcctccaagattatctcctacacccaaggtttcatgttgatgaaccaagctgccaaggactacggttggaagttgaacaatgctggtattgccttgatgtggagaggtggttgtatcatcagatccgtcttcttgggtgagatcactgctgcttacagaaagaagccagacttggaaaacttgttgctctacccattcttcaacgaagccatcaccaaggcccaaaagggatggagaacctctgtttctagagctgttgaatacggtgtcccaaccccagctttcagcactgccttggccttctacgatggtttgagatccgcacaattgccagccaacttgttgcaagcccaaagagactacttcggtgcccacaccttccaagtcttgccaggtaaggaaaactccttgttgaagaagggtgaatggatccacatcaactggaccggtaagggtggtaacgtctcctcttcatcttatgacgcttag

SEQ ID NO: 52 - DNA, 1221 bp, Candida viswanathi
  source 1..1221: mol_type = genomic DNA
atgtctttaacaccacaaggagcacagctcctttcaatcaagcagtgttcggaattaccaaaggctcgcttacccgaatacatcaagtcatccaaaagcagactatacaacgtgatatggagggcttctccaccgaccaacgtctacattgccaagaaaccgtgggaaccatcggtgcacagggcgatgattgagttcatcaaccacttgcacaaggagtatccgtcgatcaacatcatcgtgaacgaggacgtggcagaggagctccgtgaagaatacgaggacccccaggaggaaaccaggtttgacaccagcgtccaccatgtgatatacacgggaaagaaccaggacattgtggacaagaccgagttgatggttacgttaggtggggacgggacgatcttgcatggcgcgagcttgttcctgaacgtcaatgtcccacccgtgttgtcgttcgctatgggaacgttggggttcttgttgcctttcaatttcaagaacttccagctgagtttcaaagaggtttatgagagtaggagcaaggcattgcatagaaaccgattggagtgtcatgttattagaaagaacgggaatgggaaacaagacaacgatgaactgcacccaaagaaaaagtacaaggtggatcaaggacgtgttgttgatgttcctgatactgataataactcaagggagatgatccatgccatgaatgacgtcacgatccatagagccagcttaccgaacttgacatcattggacatctacattgacaacgagtttttcaccacgacgtttgccgatggtgtgatcttagccaccccaactggatcgaccgcgtattcattatcagcaggcgggtccatcacccacccagcagtgccatgcatattgctcaccccgatctgcccaagatcattatcattcagacctttgatcctccccagtctgtcagacatcatggtgaagttatctgagaacaacagaaacaacatgatcgaattgactatcgacggtatcgcacaacctgacttgcatcctggtgatgagttgcacatcacctctgaagatatcgtccccggtgctgatctttccaaaacaaacgcgaagaacgggatatggtgtgtcgcgacacatcagaaccagtgggccaaagacttgaacagcttgttagggttcaacagcctgttcagggaccagaagggtaagaagttgcatttgtaa

SEQ ID NO: 53 - DNA, 1071 bp, Kluyveromyces lactis
  source 1..1071: mol_type = genomic DNA
atgccagatatgacaaacgaatcttcttctaagccagctcaaattaacattggtatcaatggttttggtagaatcggtagattggttttgcgtgctgctttgacccacccagaagttaaggtcagattaatcaataatccatccacaacaccagaatacgctgcttatttgttcaaatacgattctactcacggcaagtatcgtggtgaagttgaatttgacgatgaacgtatcatcattcaaaatgaccatgtttccgctcatatccctttgtctcattttagagaaccagagcgtatcccatgggcttcctacaacgtcgattatgttattgactcaactggtgttttcaaggaagtcgatacagcctccagacataaaggtgtcaaaaaagttatcattactgctccatcaaagaccgctccaatgtacgtctatggtgttaaccacgttaaatacaacccattgaccgatcacgttgtctctaatgcctcctgtactaccaactgtttggctccattggttaaggctttggacgatgagttcggtatcgaagaagccttgatgacaactattcatgcaactactgcttctcaaaagactgtcgatggtacttcctctggtggtaaggactggagaggcggtagatcctgccagggaaatatcattccttcatctactggtgcagctaaggctgttggtaaaatcttgcctgaattgaatggtaagatcactggtatgtctattagagtcccaacaattaatatctctttggttgacttgacattccgtacagcaaagaaaacttcttacgatgacattatgaaggctttggaacaaagatccagatccgatatgaagggtgttttgggtgttaccaaagacgccgttgtgtcctctgacttcacatccgattcacgttcatctattgttgatgctaaggccggtattgaattgaacgaccattttttcaaggtcttgtcttggtatgataatgaatatggttactcttcaagagtggttgatttatccattttcatggctcaaaaggactttgaagctggtgtttaa

SEQ ID NO: 54 - DNA, 1530 bp, synthetic construct
  source 1..1530: mol_type = other DNA
atgagaattggcattccaagagaaagattaaccaatgaaacccgtgttgcagcaaccccaaaaacagtggaacagttgttgaaattgggttttaccgtcgctgttgagtccggcgctggtcagttggcaagttttgacgataaagcctttgtgcaagctggcgctgaaattgttgaaggtaattccgtctggcagtcagagatcattttgaaggtcaatgctccattagatgatgaaattgctttattgaatcctggtacaaccttggtgagttttatctggcctgctcagaatccagaattaatgcaaaaattggctgaacgtaacgtgaccgtgatggctatggactctgtgccacgtatctcaagagcacaatccttggacgcattgtcctctatggctaacatcgccggttatagagccattgttgaagctgcacatgaatttggtagattctttactggtcaaattactgctgccggtaaagtgccaccagcaaaagtgatggtgattggtgctggtgttgcaggtttggccgccattggcgcagcaaacagtttgggcgctattgtgcgtgcattcgacaccagaccagaagtgaaagaacaagttcaaagtatgggcgctgaatttttggagttggattttaaagaggaagctggctccggcgatggctatgccaaagtgatgtccgacgctttcatcaaagctgaaatggaattgtttgccgcccaggcaaaagaggtcgatattattgtcaccaccgctttgattccaggcaaaccagctccaaagttgattacccgtgaaatggttgactccatgaaggctggcagtgtgattgttgacttggcagcccaaaacggcggcaactgtgaatacaccgtgccaggtgaaatcttcactaccgaaaatggtgtcaaagtgattggttataccgatttgccaggccgtttgccaacccaatcctcacagttgtacggcacaaacttggttaatttgttgaaattgttgtgcaaagagaaagacggcaatatcactgttgattttgatgatgtggtgattagaggcgtgaccgtgatccgtgctggcgaaattacctggccagcaccaccaattcaggtttcagctcagccacaggctgcacaaaaagctgcaccagaagtgaaaactgaggaaaaatgtacctgctcaccatggcgtaaatacgctttgatggctttggcaatcattttgtttggctggatggcatccgttgctccaaaagaatttttgggtcacttcaccgttttcgctttggcctgcgttgtcggttattacgtggtgtggaatgtttcccacgctttgcatacaccattgatgtccgtcaccaacgctatttcaggtattattgttgtcggagcattgttgcagattggccagggcggctgggtttccttcttgagttttatcgctgtgttgattgcctccattaatattttcggtggcttcaccgtgactcagagaatgttgaaaatgttcagaaaaaat

SEQ ID NO: 55 - DNA, 1389 bp, synthetic construct
  source 1..1389: mol_type = other DNA
atgtctggaggattagttacagctgcatacattgttgccgctatcttgtttatcttcagtttggccggtttgtccaaacatgaaacctccagacagggtaacaacttcggtatcgccggtatggctattgctttaatcgcaaccatttttggaccagatactggtaatgttggctggatcttgttggctatggtcattggtggtgctattggtatccgtttggctaagaaagttgaaatgaccgaaatgccagaattggtggctatcttgcattccttcgtgggtttggctgccgtcttggttggctttaactcctatttgcatcatgacgctggaatggcaccaattttggtcaatattcacttgaccgaagtgttcttgggtatcttcatcggtgctgttaccttcactggttccgtggtggctttcggcaaattgtgtggcaagatttcctctaaaccattgatgttgccaaaccgtcacaaaatgaacttggctgctttggtcgtttccttcttgttgttgattgtttttgttagaaccgactccgtcggcttgcaagtgttggcattgttgattatgaccgctattgctttggttttcggctggcatttagtcgcctccatcggtggtgcagatatgccagtggtggtgtccatgttgaactcctactccggctgggctgccgccgctgccggctttatgttgtccaacgacttgttgattgtgactggtgctttggtcggttcttccggtgctatcttgtcttacattatgtgtaaggctatgaaccgttcctttatctccgttattgctggtggtttcggcaccgacggctcctctactggcgatgatcaggaagtgggtgagcacagagaaatcaccgcagaagagacagctgaattgttgaaaaactcccattcagtgatcattactccaggttacggcatggcagtcgctcaggctcaatatcctgtcgctgaaattactgagaaattgagagctagaggtattaatgtgcgtttcggtatccacccagtcgctggtcgtttgcctggacacatgaacgttttgttggctgaagcaaaagttccatacgacatcgtgttggaaatggacgagatcaatgatgactttgctgataccgataccgttttggtgattggtgctaacgataccgttaacccagctgctcaggatgatccaaagagtccaatcgctggtatgcctgtgttggaagtgtggaaagctcagaacgtgattgtctttaaacgttccatgaacactggctatgctggtgtgcaaaacccattgttcttcaaggaaaacacccacatgttgtttggtgacgccaaagcctccgtggatgcaatcttgaaggctttgtaa